(19) United States
(12) Patent Application Publication
(10) Pub. No.: US 2022/0392568 A1
(43) Pub. Date: Dec. 8, 2022

(54) METHOD FOR IDENTIFYING TRANSPLANT DONORS FOR A TRANSPLANT RECIPIENT

(71) Applicant: CareDx, Inc., Brisbane, CA (US)

(72) Inventors: Christopher Neal NEWBOUND, Duncraig (AU); David Charles SAYER, South Fremantle (AU)

(21) Appl. No.: 17/773,031

(22) PCT Filed: Oct. 30, 2020
(86) PCT No.: PCT/IB2020/060191

§ 371 Date: Apr. 28, 2022

(30) Foreign Application Priority Data
Oct. 31, 2019 (AU) 2019904119

(51) Int. Cl. G16B 20/00; C12Q 1/6881

(52) U.S. Cl. CPC G16B 20/00; C12Q 1/6881

(57) ABSTRACT

The present disclosure relates to a method for identifying one or more potential transplant donors for a recipient in need of a transplant, the method comprising: generating a gene dosage map for each locus of a gene complex for the one or more potential donors and the recipient; comparing the gene dosage maps of the one or more potential donors and the recipient; and determining one or more transplant donors as a transplant match for a recipient in need of a transplant if the gene dosage map of the one or more transplant donors correlates with the gene dosage map of the recipient in need of a transplant; wherein the closer the correlation between the gene dosage maps of the one or more donors compared to the recipient, the higher the probability of the one or more donors being a transplant match and/or best transplant match for the recipient.

### TECHNICAL FIELD

The present disclosure relates to a novel method for identifying one or more potential transplant donors for a recipient in need of a transplant. In particular, the present disclosure relates to methods for generating a gene dosage map of a highly polymorphic genomic region, such as the HLA gene region, for one or more potential transplant donors and the recipient to determine transplant outcome.

The present application claims priority from Australian provisional application no. 2019904119, filed on 31 Oct. 2019, the entirety of which is incorporated herein by reference.

### BACKGROUND

The major histocompatibility complex (MHC) is a group of genes found in all higher vertebrates that code for proteins found on the surfaces of cells that help the immune system recognize foreign substances. In humans, the MHC complex is also known as the human leukocyte antigen (HLA) system and is a gene dense region of approximately 4 Mb in length with more than 200 genes located close together on chromosome 6. Genes in this complex are categorized into three basic groups: class I, class II, and class III on the basis of their tissue distribution, structure, and function (Klein et al. 2000).

The Class I genes code for cell-surface glycoproteins on most nucleated cells and are involved with antigen presentation to T-cytotoxic cells. There are three main MHC class I gene loci in humans, known as HLA-A, HLA-B, and HLA-C. Class II genes code for glycoproteins expressed on antigen-presenting cells, such as macrophages, dendritic cells, and B cells, and they present antigen to T-helper cells. There are six main MHC class II loci in humans: HLA-DPA1, HLA-DPB1, HLA-DQA1, HLA-DQB1, HLA-DRA, and HLA-DRB1. Class III genes code for secreted proteins that have immunological actions, including some complement components as well as some cytokines, including tumor necrosis factor (TNF). In summary, all of these genes participate in, and control, the immune responses to pathogens and tumor surveillance. Therefore, HLA genes manifest high structural polymorphism, meaning, the HLA genes have many possible variations (alleles), allowing each person's immune system to react to a wide range of foreign invaders. The polymorphism of HLA genes is so high that in a mixed population (non-endogamic) there are not two individuals with exactly the same set of MHC genes and molecules, with the exception of identical twins (Guild et al. 1955).

High polymorphism of the HLA genes against different HLA antigens represent a major barrier to tissue or organ transplantation because, for example, a recipient's immune response may recognise molecules (HLA antigens) expressed on the surface of a donor's transplanted tissue cells or organ cells as being ‘non-self’ leading to rejection or transplant failure (Garcia et al. 2012, Sheldon, S. and Poulton, K. 2006, and Mandi, 2013). Acceptance or rejection of the graft after tissue transplantation is primarily determined by compatibility of HLA gene sequences between donor and recipient. These responses may be extreme such as in the case of graft vs host disease (GVHD) mediated by alloreactive cytotoxic T-lymphocytes (CTL) after allogeneic HSC transplantation, or in the case of acute rejection mediated by preformed anti-HLA specific antibodies after tissue or organ transplantation. Therefore, precise HLA typing is of great clinical importance having important consequences on graft and transplantation outcomes, and a great deal of research effort has been devoted to the identification of HLA subtypes and development of typing methods.

Major advancements have been made for HLA typing using DNA-based HLA typing methods utilising molecular techniques such as: sequence-specific oligonucleotide probe hybridization (SSOP); Sanger sequencing-based typing (SBT) methods; sequence-specific primer amplification (SSP); sequencing-based typing (SBT); reference strand-based conformation analysis (RSCA); short tandem repeat (STR) genotyping; and the use of next-generation sequencing data. Whilst these newer typing methods have significantly improved HLA typing resolution, these typing methods possess several limitations, such as time-consuming protocols, low throughput, unphased data, ambiguity and obtained results containing errors owing to artefact amplification (such as for example, artefacts owing to substitutions and PCR-chimeras) during PCR or indels during the sequencing process. As such, precise HLA typing to ensure good transplant between a donor and a recipient outcome remains very challenging owing to the high degree of polymorphism among HLA genes, discerning true alleles versus sequencing errors, sequence similarity among these genes, and extreme linkage disequilibrium of the locus.

Thus, there remains a need for an improved method for identifying and determining one or more suitable transplant donors for a recipient in need of a transplant.

### SUMMARY

The present inventors have, for the first time, demonstrated the use of a novel method for identifying one or more transplant donors for a recipient in need of a transplant. As described herein, the inventors have demonstrated the use of a hybrid-capture next generation sequencing (NGS) technique and the utilisation of a sequencing alignment software to generate a gene dosage map based on gene copy number for genes in highly polymorphic gene blocks or complexes, such as the MHC gamma block or HLA gene complex. The inventors have demonstrated that the gene dosage map of HLA genes are vastly different for two individuals, for which the two individuals may have been previously determined to be a good transplant match using techniques known in the art, for example, genotyping based polymerase chain reaction (PCR) and DNA sequencing. This finding provides the basis for a novel method of analysing and interpreting sequence reads via gene-specific locus allocations, which provides a means of augmenting current sequence typing methodologies, to make a more an improved determination of transplant outcome between one or more transplant donors and a recipient in need of a transplant.

In a first aspect, the present disclosure provides a method for identifying one or more potential transplant donors for a recipient in need of a transplant, the method comprising:
a) generating a gene dosage map for each locus of a gene complex for the one or more potential donors and the recipient;
b) comparing the gene dosage maps of the one or more potential donors and the recipient; and
c) determining one or more transplant donors as a transplant match for a recipient in need of a transplant if the gene dosage map of the one or more transplant donors correlates with the gene dosage map of the recipient in need of a transplant;
   wherein the closer the correlation between the gene dosage maps of the one or more donors compared to the recipient, the higher the probability of the one or more donors being a transplant match and/or best transplant match for the recipient.

In a second aspect, the present disclosure provides a method for identifying one or more potential transplant donors for a recipient in need of a transplant, the method comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each of the locus of the gene complex determined in step (c); and
e) comparing the generated gene dosage map of the one or more potential transplant donors with the generated gene dosage map of the recipient;
   wherein the one or more potential transplant donors is identified as a transplant match and/or best transplant match for a recipient in need of a transplant if the gene dosage map of the one or more transplant donors correlates with the gene dosage map of the recipient.

In one embodiment of the first and second aspects, the transplant is a graft and/or tissue and/or organ transplant. In one embodiment of the first and second aspects, the method reduces the likelihood of the transplant recipient developing graft versus host disease (GVHD). In one embodiment of the first and second aspects, the method prevents the likelihood of the transplant recipient developing graft versus host disease (GVHD). In one embodiment of the first and second aspects, the method reduces the likelihood of graft and/or tissue and/or organ transplant rejection. In one embodiment of the first and second aspects, the transplant is any type of transplant where transplant phenotype is observed based on sequence and/or gene copy number differences.

In a third aspect, the present disclosure provides a method for reducing the likelihood of a transplant recipient developing graft versus host disease (GVHD), the method comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors.

In a fourth aspect, the present disclosure provides a method for reducing the likelihood of any transplant rejection where transplant phenotype is observed based on gene and/or sequence copy number differences, the method comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of any transplant rejection where transplant phenotype is observed based on gene and/or sequence copy number differences following transplantation of a graft from the one or more transplant donors.

In one embodiment of the fourth aspect, the method reduces the likelihood of a transplant recipient developing transplant rejection. In one embodiment of the fourth aspect, the method reduces the likelihood of a transplant recipient developing graft and/or tissue and/or organ rejection.

In a fifth aspect, the present disclosure provides a method for analysing sequences to identify one or more potential transplant donors for a recipient in need of a transplant, the method comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each of the locus of the gene complex determined in step (c); and
e) comparing the generated gene dosage map of the one or more potential transplant donors with the generated gene dosage map of the recipient;
   wherein the one or more potential transplant donors is identified as a transplant match and/or best transplant match for a recipient in need of a transplant, if the gene dosage map of the one or more transplant donors correlates with the gene dosage map of the recipient.

In one embodiment of the fifth aspect, the transplant is a graft and/or tissue and/or organ transplant. In one embodiment of the fifth aspect, the transplant donors are identified to reduce the likelihood of the transplant recipient developing graft versus host disease (GVHD). In one embodiment of the fourth aspect, the transplant is any type of transplant where transplant phenotype is observed based on sequence and/or gene copy number differences.

In a sixth aspect, the present disclosure provides a method of preventing graft versus host disease (GVHD) disease between one or more potential transplant donors and a recipient comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of the transplant recipient developing graft versus host disease following transplantation of a graft and/or tissue and/or organ from the one or more transplant donors, and

selecting graft and/or tissue and/or organ from a transplant donor having a gene dosage map that correlates with the gene dosage map of the recipient for transplant to the recipient.

In a seventh aspect, the present disclosure provides a method of preventing any transplant rejection where transplant phenotype is observed based on sequence and/or gene copy number differences comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of any transplant rejection where transplant phenotype is observed based on sequence and/or gene copy number differences following transplantation of a graft and/or tissue and/or organ from the one or more transplant donors, and selecting graft and/or tissue and/or organ from a transplant donor having a gene dosage map that correlates with the gene dosage map of the recipient for transplant to the recipient.

In an eighth aspect, the present disclosure provides a method of transplanting tissue from one or more potential transplant donors to a recipient, comprising:
(i) identifying one or more potential transplant donors for a recipient in need of a transplant comprising the steps of:
   a) generating sequences of a gene complex from a nucleic acid sample obtained from the one or more potential transplant donors and the recipient;
   b) assigning a plurality of the sequences generated in step (a) corresponding to each locus of the gene complex;
   c) determining gene dosage for each locus of the gene complex from the plurality of sequences assigned in step (b);
   d) generating a gene dosage map of the gene complex for the one or more potential transplant donors and the recipient from the gene dosage for each locus of the gene complex determined in step (c); and
   e) comparing the generated gene dosage map of the one or more potential transplant donors with the generated gene dosage map of the recipient;
      wherein the gene dosage map of the one or more potential transplant donors correlates with the gene dosage map of the recipient in need of a transplant is indicative of reduced likelihood of the transplant recipient developing graft versus host disease following transplantation of a graft and/or tissue and/or organ from the one or more transplant donors, and
(ii) transplanting graft and/or tissue and/or organ from a transplant donor having a gene dosage map that correlates with the gene dosage map of the recipient to the recipient.

In a ninth aspect the present disclosure provides a method of transplanting a graft and/or tissue and/or organ from one or more potential transplant donors to a recipient, comprising:
(i) identifying one or more potential transplant donors for a recipient in need of a transplant comprising the steps of:
   a) generating sequences of a gene complex from a nucleic acid sample obtained from the one or more potential transplant donors and the recipient;
   b) assigning a plurality of the sequences generated in step (a) corresponding to each locus of the gene complex;
   c) determining gene dosage for each locus of the gene complex from the plurality of sequences assigned in step (b);
   d) generating a gene dosage map of the gene complex for the one or more potential transplant donors and the recipient from the gene dosage for each locus of the gene complex determined in step (c); and
   e) comparing the generated gene dosage map of the one or more potential transplant donors with the generated gene dosage map of the recipient;
      wherein the gene dosage map of the one or more potential transplant donors correlates with the gene dosage map of the recipient in need of a transplant is indicative of reduced likelihood of the transplant recipient developing graft and/or tissue and/or organ rejection following transplantation of a graft and/or tissue and/or organ from the one or more transplant donors, and
(ii) transplanting graft and/or tissue and/or organ from a transplant donor having a gene dosage map that correlates with the gene dosage map of the recipient to the recipient.

In one embodiment of the ninth aspect, the present disclosure provides a method of transplanting a transplant whose transplant phenotype is observed based on gene and/or sequence copy number differences. In one embodiment of the ninth aspect, the gene dosage map of the one or more potential transplant donors correlates with the gene dosage map of the recipient in need of a transplant is indicative of reduced likelihood of the transplant recipient developing graft versus host disease (GVHD). In one embodiment of the ninth aspect, the gene dosage map of the one or more potential transplant donors correlates with the gene dosage map of the recipient in need of a transplant is indicative of reduced likelihood of the transplant recipient developing transplant rejection for any transplant whose phenotype is observed based on gene and/or sequence copy number differences. In one embodiment of the ninth aspect, the gene dosage map of the one or more potential transplant donors correlates with the gene dosage map of the recipient in need of a transplant is indicative of reduced likelihood of the transplant recipient developing graft and/or tissue and/or organ rejection.

In one embodiment, generating the gene dosage map for each locus of the gene complex for the one or more potential donors and the recipient comprises dividing the plurality of sequences assigned to each locus by the plurality of sequences assigned to all loci of the gene complex.

In one embodiment, the gene dosage for each locus is copy number for each locus of the gene complex.

In one embodiment, the gene dosage map is the copy number for all loci of the gene complex.

In one embodiment, the copy number of each locus and all loci of the gene complex allows determination of zygosity for each locus and all loci of the gene complex. In one embodiment, the copy number of sequences allows determination of zygosity for each locus and all loci of the gene complex.

In one embodiment, the copy number of each locus and loci of the gene complex allows determination of whether two alleles have an identical sequence. In one embodiment, the copy number of sequences allows determination of whether two alleles have an identical sequence

In one embodiment, the gene complex is a highly polymorphic gene complex.

In one embodiment, the gene complex is a gene complex pertaining to transplantation.

In one embodiment, the highly polymorphic gene complex is an HLA gene complex. In one embodiment, the highly polymorphic gene complex is the MHC gamma block. In one embodiment, the highly polymorphic gene complex is MR gene complex. In one embodiment, the highly polymorphic gene complex is Rhesus gene complex. In one embodiment, the highly polymorphic gene complex may be any gene complex relating to any transplant where transplant phenotype is observed based on gene and/or sequence copy number differences.

In one embodiment, step (b) of the method of the present disclosure comprises assigning a plurality of the sequences generated in step (a) corresponding to each locus of the gene complex based on: one or more regions of each locus; all exons in each locus; and/or an entire sequence of each locus.

In one embodiment, step (b) of the method of the present disclosure comprises assigning a plurality of the sequences generated in step (a) using a computer program.

In one embodiment, the computer program is a sequence editing and alignment program.

In a tenth aspect, the present disclosure provides a method wherein generating sequences of a gene complex from a nucleic acid sample obtained from the one or more potential transplant donors and the recipient, is a method for identifying gene alleles in the one or more transplant donors and the recipient in need of a transplant, the method comprising:
a) contacting a nucleic acid sample from the one or more transplant donors and the recipient with oligonucleotide probes, wherein the oligonucleotide probes hybridize to gene target sequences in the nucleic acid sample;
b) enriching a nucleic acid by hybridizing the nucleic acid to one or more oligonucleotide probes;
c) separating nucleic acid hybridized to the one or more oligonucleotide probes from nucleic acid not hybridized to the one or more oligonucleotide probes; and
d) sequencing the enriched nucleic acid to identify one or more gene alleles; wherein the gene target sequences are in a non-coding region of the gene.

In one embodiment, the gene is a highly polymorphic gene.

In one embodiment, the gene is a gene pertaining to transplantation.

In one embodiment, the highly polymorphic gene is an HLA gene. In one embodiment, the highly polymorphic gene complex is the MHC gamma block. In one embodiment, the highly polymorphic gene complex is MR gene complex. In one embodiment, the highly polymorphic gene complex is Rhesus gene complex. In one embodiment, the highly polymorphic gene complex may be any gene complex relating to any transplant where transplant phenotype is observed based on gene and/or sequence copy number differences.

In one embodiment, the method comprises amplifying the nucleic acid bound to the one or more oligonucleotide probes. In one embodiment, the method comprises sequencing an HLA gene exon, or any gene exon pertaining to transplantation.

In one embodiment, the method comprises sequencing an entire HLA gene, or an entire gene pertaining to transplantation. In another aspect, the HLA gene or the gene pertaining to transplantation may be sequenced in part or in its entirety.

In one embodiment, the one or more oligonucleotide probes comprises a capture tag.

In one embodiment, the capture tag is biotin or streptavidin.

In one embodiment, the method further comprises contacting the capture tag with a binding agent.

In one embodiment, the binding agent is biotin or streptavidin.

In one embodiment, the nucleic acid sample from the one or more transplant donors and the recipient in need of a transplant that is contacted with the one or more oligonucleotide probes comprises single stranded nucleic acid.

In one embodiment, the nucleic acid sample is fragmented before being contacted with the one or more oligonucleotide probes.

In one embodiment, the nucleic acid sample is fragmented after being contacted with the one or more oligonucleotide probes.

In one embodiment, the fragments of the nucleic acid sample have an average length greater than about 100 bp.

In one embodiment, the nucleic acid sample from the one or more transplant donors and the recipient in need of a transplant is genomic DNA extracted from a biological sample.

In one embodiment, the biological sample is anti-coagulated whole blood.

In one embodiment, the genomic DNA is at a concentration of about 10 ng/μl to about 100 ng/μl.

In one embodiment, sequencing is performed using high-throughput sequencing. In the present disclosure, sequencing of the gene complex is performed using high-throughput sequencing. In the present disclosure, sequencing of the HLA gene exon or the exon of any gene pertaining to transplantation is performed using high-throughput sequencing. In the present disclosure, sequencing of the entire HLA gene or any gene pertaining to transplantation is performed using high-throughput sequencing.

In one embodiment, the high-throughput sequencing is hybrid-capture next generation sequencing technique.

In one embodiment, the sequences are generated in a computer readable form. In one embodiment, the sequences are gene sequences. In one embodiment, the sequences are intergenic sequences. In another embodiment, the sequences are gene sequences and intergenic sequences.

In one embodiment, the computer readable form is FASTQ.

In an eleventh aspect, the present disclosure provides a kit for identifying one or more potential transplant donors for a recipient in need of a transplant, the kit comprising:
a) one or more nucleic acid reagents to prepare a nucleic acid library from a nucleic acid sample; and
b) one or more oligonucleotide probes that hybridise to gene target sequences of the nucleic acid sample.

In one embodiment of the eleventh aspect, the transplant donors are identified to reduce the likelihood of developing graft versus host disease (GVHD). In one embodiment of the eleventh aspect, the transplant donors are identified to reduce the likelihood of developing graft and/or tissue and/or organ rejection. In one embodiment of the eleventh aspect, the transplant is any type of transplant where transplant phenotype is observed based on gene and/or sequence copy number differences.

A twelfth aspect provides a kit when used according to the method of any one of the preceding aspects for identifying one or more potential transplant donors for a recipient in need of a transplant, the kit comprising:

a) one or more nucleic acid reagents to prepare a nucleic acid library from a nucleic acid sample; and
b) one or more oligonucleotide probes that hybridise to gene target sequences of the nucleic acid sample.

A thirteenth aspect provides use of a kit according to the method of any one of the preceding aspects for identifying one or more potential transplant donors for a recipient in need of a transplant, the kit comprising:

a) one or more nucleic acid reagents to prepare a nucleic acid library from a nucleic acid sample; and
b) one or more oligonucleotide probes that hybridise to gene target sequences of the nucleic acid sample.

In one embodiment of the twelfth or thirteenth aspects, the transplant is a graft and/or tissue and/or organ transplant. In one embodiment of the twelfth or thirteenth aspects, the transplant is a transplant relating to developing graft versus host disease (GVHD). In one embodiment of the twelfth or thirteenth aspects, the transplant is any type of transplant where transplant phenotype is observed based on gene and/or sequence copy number differences.

In one embodiment, the nucleic acid sample is genomic DNA.

In one embodiment, the one or more nucleic acid reagents to prepare a nucleic acid library comprises one or more reagents to bind to the genomic DNA, one or more reagents to fragment the genomic DNA and one or more reagents to tag the genomic DNA to beads.

In one embodiment, the gene target sequences are sequences for a highly polymorphic gene complex.

In one embodiment, the polymorphic gene complex is a polymorphic gene complex pertaining to transplantation.

In one embodiment, the polymorphic gene complex is a HLA gene complex.

In one embodiment, the one or more oligonucleotide probes comprises a capture tag.

In one embodiment, the capture tag is biotin or streptavidin

In one embodiment, the kit further comprises a binding agent

In one embodiment, the binding agent is biotin or streptavidin.

In one embodiment, the binding agent is coupled to a substrate.

In one embodiment, the substrate is a magnetic substrate.

A fourteenth aspect, the present disclosure provides a kit comprising one or more nucleic acid reagents to perform sequencing of a nucleic acid library using the method of the tenth aspect, wherein sequencing reads are generated in a computer readable form.

In one embodiment, the sequencing reads are next generation sequencing (NGS) reads. In one embodiment the next generation sequencing (NGS) reads are gene sequences. In one embodiment the next generation sequencing (NGS) reads are intergenic sequences. In one embodiment the next generation sequencing (NGS) reads are gene sequences and intergenic sequences.

In one embodiment, the kit of the eleventh to fourteenth aspects further comprises a computer program to analyse and edit the NGS reads and generate a gene dosage map for each locus of a gene complex using the method of any one of the first to tenth aspects, wherein one or more potential transplant donors is identified as a transplant match and/or best transplant match for a recipient in need of a transplant.

In one embodiment, the computer program is a sequence editing and alignment program. In one embodiment the computer program is a sequence editing and alignment program is Assign™ TruSight version 2.1 software (“Assign” software) by CareDx Inc. In another embodiment, the sequence editing and alignment software program is the AlloSeq Assign software by CareDx.

In a fifteenth aspect, the present disclosure provides a gene dosage map for each locus of a gene complex for one or more potential donors and a recipient generated using the method of any one of the first to tenth aspects.

A sixteenth aspect provides use of a gene dosage map for each locus of a gene complex for one or more potential donors and a recipient generated using the methods of any one of the first to tenth aspects for:
a) identifying one or more potential transplant donors for a recipient in need of a transplant;
b) reducing the likelihood of a transplant recipient developing graft versus host disease (GVHD);
c) treating graft versus host disease (GVHD) disease between one or more potential transplant donors and a recipient;
d) determining gene copy number difference; and
e) determining zygosity for each locus and all loci of the gene complex.

In one embodiment, the gene copy number difference may be 0 or may be 1 or may be 2 or may be 3. In one embodiment, gene copy number difference may be more than 3. In one embodiment, the copy number difference is copy number variation which is indicative of chromosomal rearrangement. In one embodiment, chromosomal rearrangement occurs by homologous recombination mechanism. In one embodiment, chromosomal rearrangement occurs by non-homologous recombination mechanism.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 is a representative schematic of the total number of sequence reads generated using hybrid-capture next generation sequencing (NGS) for a first patient i.e. patient 1. The 250,000 sequences represent the total number of sequences generated for all HLA genomic regions which have been hybridized to by HLA target-specific biotinylated oligonucleotide probes. Of the total 250,000 reads, these reads are analysed, edited and compared to a reference genome which comprises a library of sequences of HLA alleles, using the Assign™ TruSight version 2.1 software (“Assign” software). The consensus regions of the total reads are analysed and assigned by the Assign software into HLA gene specific allocations, namely, Gene A (with 27,000 assigned reads), Gene B (with 25,000 assigned reads) and Gene C (with 30,000 assigned reads) respectively.

FIG. 2 is a representative schematic of the total number of sequence reads generated using hybrid-capture NGS for a second patient i.e. patient 2. From FIG. 2, a total of 220,000 reads was generated. Of the total 220,000 reads for patient 2, there are 24,000 assigned reads for Gene A, 11,000 assigned reads for Gene B and 26,000 assigned reads for Gene C.

FIG. 3 provides an example of how gene dosage for all loci of a gene complex (the MHC gene complex) is calculated using the number of sequence reads to generate a gene dosage map of the gene complex. Column A denotes samples from twenty different patients. Column B denotes the total NGS reads for each patient. Column C denotes the assigned reads for all HLA gene loci and column D denotes assigned reads specifically to a particular locus, the HLA-H gene. Column E represents the proportion of reads at a particular locus (i.e. the HLA-H gene) relative to all gene loci in a sample as a ratio of the means proportion. The values in Column E for the HLA-H gene are obtained by dividing the number of assigned sequences for the HLA-H gene by the number of assigned sequences for all gene loci. Column F denotes the values in column E converted to a percentage proportion.

FIG. 4 is a graphical representation that it has been observed in several individuals that several individuals (shown by arrows) are seen to have a reduction in sequence reads for the HLA-H locus compared to total sequence reads, which may be quantitatively demonstrated via a ratio of the two measures (see column F of FIG. 3 and FIG. 5).

FIG. 5 is a graphical representation of the calculated percentage proportion for the HLA-H gene for 20 patients from column F of FIG. 3.

FIG. 6 is a gene dosage map of the HLA gene complex generated for a first patient i.e. patient 1. The gene dosage map is a representation of gene dosage for all gene loci.

FIG. 7 is a gene dosage map of the HLA gene complex generated for a second patient i.e. patient 2.

FIG. 8 is a gene dosage map of the HLA gene complex generated for a third patient i.e. patient 3.

FIG. 9 is a graphical representation of the percentage proportion of HLA genes: HLA-A; HLA-B and HLA-C in 18 samples, whereby the sequences were generated using PCR-based methodology and not using the hybrid-capture NGS sequencing technique. The percentage proportion for each of the HLA-genes was calculated using the method disclosed in the present disclosure. Sequences generated using PCR-based methodology is not an ideal method for determining gene dosage because exponential propagation of DNA from a sample will result in decreased uniformity between loci and patient samples. In the present disclosure, the use of hybrid-capture NGS technique allows for comparison using the same concentrations of DNA and the sequence reads can be adjusted using total sequence reads.

FIG. 10 shows the gene dosage map generated via the method of the present disclosure for two patients which had poor transplant outcomes. As shown in FIG. 10, the gene dosage map informs that the gene dosage maps of the two individuals are different.

FIG. 11 shows the gene dosage map generated via the method of the present disclosure for a first pair of clinical samples #105 and #116. As shown in FIG. 11, the gene dosage map of these two clinical samples are similar.

FIG. 12 shows the gene dosage map generated via the method of the present disclosure for a second pair of clinical samples #107 and #104. As shown in FIG. 12, the gene dosage map of these two clinical samples are similar.

### DETAILED DESCRIPTION

### General Techniques and Definitions

Unless specifically defined otherwise, all technical and scientific terms used herein shall be taken to have the same meaning as commonly understood by one of ordinary skill in the art.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The present disclosure is not to be limited in scope by the specific examples described herein, which are intended for the purpose of exemplification only. Functionally equivalent products, compositions and methods are clearly within the scope of the disclosure, as described herein.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings and for either meaning.

As used herein, the term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

The term “gene” refers to a nucleic acid (e.g., DNA or RNA) sequence that comprises coding sequences necessary for the production of an RNA or a polypeptide or its precursor. The fragments may range in size from a few nucleotides to the entire gene sequence minus one nucleotide. Thus, “a nucleotide comprising at least a portion of a gene” may comprise fragments of the gene or the entire gene. The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

As used herein, an “allele” refers to an alternative sequence at a particular locus. The length of an allele can be as small as 1 nucleotide base, but is typically larger. Allelic sequence can be amino acid sequence or nucleic acid sequence.

As used herein, a “locus” is the singular of “loci” and is a short sequence that is usually unique and usually found at one particular location in the genome by a point of reference e.g., a short DNA sequence that is a gene, or part of a gene or intergenic region. In some embodiments, a locus is a unique PCR product at a particular location in the genome. Loci is the plural of “locus” and may comprise one or more polymorphisms; i.e., alternative alleles present in some individuals. As used herein, ‘locus’ may refer to gene complex locus, such as the HLA complex locus, which is a genomic segment of the chromosome that contains a cluster of genes. The complex locus may contain a cluster of gene loci.

Thus, the terms “variant” and “mutant” when used in reference to a nucleotide sequence refer to a nucleic acid sequence that differs by one or more nucleotides from another, usually related nucleotide acid sequence. A “variation” is a difference between two different nucleotide sequences typically, one sequence is a reference sequence.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. When present in a DNA form, the oligonucleotide may be single-stranded (i.e., the sense strand) or double-stranded.

The term “polymorphism” refers to the occurrence of two or more alternative genomic sequences or alleles between or among different genomes or individuals. The variation may comprise but is not limited to one or more base changes, the insertion of one or more nucleotides or the deletion of one or more nucleotides. A polymorphism includes a single nucleotide polymorphism (SNP), a simple sequence repeat (SSR) and indels, which are insertions and deletions. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the later may be associated with rare but important phenotypic variation. In some embodiments, a “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (indels), simple sequence repeats of DNA sequence (SSRs) a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms.

The term “polymorphic” refers to the condition in which two or more variants of a specific genomic sequence are found in a population.

The term “polymorphic site” is the locus at which the variation occurs. A polymorphic site generally has at least two alleles, each occurring at a significant frequency in a selected population. A polymorphic locus may be as small as one base pair, in which case it is referred to as single nucleotide polymorphism (SNP). The first identified allelic form is arbitrarily designated as the reference, wild-type, common or major form, and other allelic forms are designated as alternative, minor, rare or variant alleles.

The term “genotype” refers to a description of the alleles of a gene contained in an individual or sample. The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

As used herein, “phenotype” means the detectable characteristics of a cell or organism which are a manifestation of gene expression.

The term “gene dosage” used herein refers to the number of copies of a particular gene present in a genome. As described herein, “gene dosage” refers to the number of copies of gene loci in a locus of a gene complex, for example, the HLA gene complex locus. As described herein, “gene dosage” may refer to the number of copies of one or more gene loci or all gene loci in a locus of a gene complex, for example, one or more gene loci or all gene loci in the HLA gene complex locus.

The term “gene dosage map” refers to a pictorial showing the relative amounts of each and every loci of a gene complex relative to each other. The relative amounts of each and every gene locus is the copy number of each and every gene locus of a gene complex relative to each other.

The term “gene copy number” or “copy number variation” is a phenomenon in which sections of the genome are repeated and the number of repeats in the genome varies between individuals in the human population. The term “copy number variation” includes an intermediate-scale genetic change, operationally defined as segments greater than 1,000 base pairs in length but typically less than 5 megabases, which is the cytogenetic level of resolution. Copy number variations (CNVs) include both additional copies of sequence (duplications) and losses of genetic material (deletions). As described herein, there may be a difference in the copy number for any gene complex, or highly polymorphic gene complex or a gene complex relating to transplantation or any gene complex associated with a transplant whose transplant phenotype is based on copy number differences. Copy number variation may be observed in gene copy number differences and/or in sequences. As described herein, there may a difference in the copy number of HLA genes in the genome of an individual. In one embodiment, the gene copy number difference measured may be 0 or may be 1 or may be 2 or may be 3 or may be more than 3.

The term “zygosity” refers to the degree of genetic similarity of the alleles for a trait in an organism. Most eukaryotes have two matching sets of chromosomes and are termed as being diploid. Diploid organisms have the same loci on each of their two sets of homologous chromosomes except that the sequences at these loci may differ between the two chromosomes in a matching pair and that a few chromosomes may be mismatched as part of a chromosomal sex-determination system. If both alleles of a diploid organism are the same, the organism is homozygous at that locus. If both alleles are different, the organism is heterozygous at that locus. If one allele is missing, it is hemizygous, and, if both alleles are missing, it is nullizygous.

As used herein, “typing” refers to any method whereby the specific allelic form of a given HLA genomic polymorphism is determined. For example, a single nucleotide polymorphism (SNP) is typed by determining which nucleotide is present (e.g., an A, G, T, or C). Insertion/deletions (indels) are determined by determining if the indel is present. Indels can be typed by a variety of assays including, but not limited to, marker assays.

As used herein, “genotyping” refers to any technology that detects small genetic differences that can lead to major changes in phenotype, including both physical differences that make us unique and pathological changes underlying disease.

The term nucleic acid” or nucleic acid sequence” or nucleic acid molecule” refers to deoxyribonucleotides or ribonucleotides and polymers thereof in either single- or double stranded form. The term nucleic acid is used interchangeably with gene, complementary DNA (cDNA), messenger RNA (mRNA), oligonucleotide, and polynucleotide.

As used herein, the terms “transplant” or “transplanting” refer to the grafting or introduction of tissue or cells obtained from one individual (the donor) into or onto the body of another individual (the recipient). The cells or tissue that are removed from the donor and transplanted into the recipient are referred to as a “graft”. Examples of tissues commonly transplanted are bone marrow, hematopoietic stem cells, organs such as liver heart, skin, bladder, lung, kidney, cornea, pancreas, pancreatic islets, brain tissue, bone, and intestine. In one embodiment, the transplant is a tissue transplant. In another embodiment, the transplant is an organ transplant. In yet another embodiment, the transplant is a hematopoietic stem cell transplant.

The person skilled in the art, would understand that the term “haplotype” refers to a combination of alleles that are located closely, or at adjacent loci, on a chromosome and that are inherited together, or a set of single nucleotide polymorphisms on a single chromosome of a chromosome pair that are statistically associated.

The term “subject” refers to any animal having a disease which requires treatment by the present method. In addition to primates, such as humans, a variety of other mammals can be treated using the methods of the present invention. For instance, mammals including, but not limited to, cows, sheep, goats, horses, dogs, cats, guinea pigs, rats or other bovine, ovine, equine, canine, feline, rodent or murine species can be treated.

The person skilled in the art, would understand that the term “haplotype” refers to a combination of alleles that are located closely, or at adjacent loci, on a chromosome and that the alleles are inherited together, or a set of single nucleotide polymorphisms on a single chromosome of a chromosome pair that are statistically associated.

The terms “HLA” and “MHC” may be used interchangeably throughout the specification but it will be understood that the terms “HLA” and “MHC” both refer to human version of the gene complex encoding the major histocompatibility complex (MHC) proteins.

### Method for Identifying One or More Transplant Donors for a Recipient

The present inventors have developed a novel method of identifying one or more transplant donors for a recipient in need of transplant. The method may be used to identify one or more transplant donors for a recipient in need of a graft transplant and/or tissue transplant, an organ transplant and/or stem cell transplant and/or any transplant whose transplant phenotype is based on sequence copy number difference. The method of the present disclosure comprises generating a gene dosage map for each loci of a gene complex for the one or more potential donors and the recipient. The generated gene dosage maps of the one or more potential donors and the recipient are compared. One or more transplant donors may be determined to be a suitable transplant match and/or the best transplant match, for a recipient in need of a transplant based on the correlation of their respective gene dosage maps.

The method developed by the inventors disclosed herein be used for identifying one or more potential transplant donors for a recipient in need of a transplant, the method comprising:

a) generating a gene dosage map for each locus of a gene complex for the one or more potential donors and the recipient;
b) comparing the gene dosage maps of the one or more potential donors and the recipient; and
c) determining one or more transplant donors as a transplant match for a recipient in need of a transplant if the gene dosage map of the one or more transplant donors correlates with the gene dosage map of the recipient in need of a transplant;
wherein the closer the correlation between the gene dosage maps of the one or more donors compared to the recipient, the higher the probability of the one or more donors being a transplant match and/or best transplant match for the recipient.

The method developed by the inventors disclosed herein be used for identifying one or more potential transplant donors for a recipient in need of a transplant, the method comprising:

a) generating a gene dosage map for each locus of HLA gene complex for the one or more potential donors and the recipient;
b) comparing the HLA complex gene dosage maps of the one or more potential donors and the recipient; and
c) determining one or more transplant donors as a transplant match for a recipient in need of a transplant if the HLA complex gene dosage map of the one or more transplant donors correlates with the gene dosage map of the recipient in need of a transplant;
wherein the closer the correlation between the HLA complex gene dosage maps of the one or more donors compared to the recipient, the higher the probability of the one or more donors being a transplant match and/or best transplant match for the recipient.

In one embodiment, the method identifies a transplant donor in which the likelihood of the recipient developing graft versus host disease (GVHD) is reduced. In another embodiment, the transplant is for any type of transplant where transplant phenotype is observed based on sequence copy number differences.

‘Transplant match’ refers the correlation between the gene dosage maps of the one or more donors compared to the recipient. The closer the correlation between the gene dosage maps of the one or more donors compared to the recipient, the higher the probability of the one or more donors being a good transplant match and/or or the best transplant match for the recipient. If the gene dosage map of the one or more transplant donors correlates with the gene dosage map of the recipient in need of a transplant, the one or more donors will be determined to be a suitable transplant match and/or good transplant match for the recipient. Correlation of gene dosage maps between one or more donors compared to the recipient, may refer to the gene dosage map comprising the gene dosage for all or nearly all gene loci in a gene complex, between the one or more transplant donors and the transplant recipient, being the same, similar or nearly similar.

‘Best transplant match’ refers to a situation where one or more transplant donors have been determined to be a suitable transplant match for the recipient, the donor determined to have a gene dosage map with the highest correlation or highest similarity with the gene dosage map of the recipient will be selected for transplant. The terms “correlate”, “correlation” and “correlating” used herein refers to the similarity of the gene dosage map between the one or more donors when compared to the gene dosage map of the recipient in need of a transplant. Particularly, the terms “correlate”, “correlation” and “correlating” all refer to the similarity in the calculated gene dosage map based on copy number of each and every loci of a gene complex. The gene dosage map of a first subject is said to correlate with the gene dosage map of a second subject if the calculated gene dosage map based on copy number of each and every loci of a gene complex is the same or is similar, to the calculated gene dosage based on copy number of each and every loci of a gene complex of the second subject.

The method developed by the inventors disclosed herein may be used for identifying one or more potential transplant donors for a recipient in need of a transplant, the method comprising generating sequences of a gene complex from a nucleic acid sample obtained from the one or more potential transplant donors and the recipient, assigning a plurality of the generated sequences corresponding to each locus of the gene complex, determining gene dosage for each locus of the gene complex from the plurality of assigned sequences and generating a gene dosage map of the gene complex for the one or more potential transplant donors and the recipient from the gene dosage determined for each loci of the gene complex, and comparing the generated gene dosage map of the one or more potential transplant donors with the generated gene dosage map of the recipient, wherein the one or more potential transplant donors is identified as a transplant match and/or best transplant match for a recipient in need of a transplant if the gene dosage map of the one or more transplant donors correlates with the gene dosage map of the recipient.

The method developed by the inventors disclosed herein may be used for identifying one or more potential transplant donors for a recipient in need of a transplant, the method comprising:

a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each of the locus of the gene complex determined in step (c); and
e) comparing the generated gene dosage map of the one or more potential transplant donors with the generated gene dosage map of the recipient;
wherein the one or more potential transplant donors is identified as a transplant match and/or best transplant match for a recipient in need of a transplant if the gene dosage map of the one or more transplant donors correlates with the gene dosage map of the recipient.

The method developed by the inventors disclosed herein may be used for identifying one or more potential transplant donors for a recipient in need of a transplant, the method comprising:

a) generating sequences of a HLA gene complex from a nucleic acid sample obtained from the one or more potential transplant donors and the recipient;
b) assigning a plurality of the sequences of HLA gene complex generated in step (a) corresponding to each locus of the HLA gene complex;
c) determining gene dosage for each locus of the HLA gene complex from the plurality of sequences assigned in step (b);
d) generating a gene dosage map of the HLA gene complex for the one or more potential transplant donors and the recipient from the gene dosage for each of the locus of the HLA gene complex determined in step (c); and
e) comparing the generated HLA complex gene dosage map of the one or more potential transplant donors with the generated gene dosage map of the recipient;
wherein the one or more potential transplant donors is identified as a transplant match and/or best transplant match for a recipient in need of a transplant if the gene dosage map of the HLA gene complex of the one or more transplant donors correlates with the gene dosage map of the HLA complex of the recipient, selecting tissue from a transplant donor having a gene dosage map of the HLA gene complex that correlates with the gene dosage map of the HLA gene complex of the recipient for transplant to the recipient.

The method developed by the inventors disclosed herein may be used for identifying one or more potential transplant donors for a recipient in need of a transplant, the method comprising:

a) generating sequences of a gene complex from a nucleic acid sample from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each of the locus of the gene complex determined in step (c); and
e) comparing the generated gene dosage map of the one or more potential transplant donors with the generated gene dosage map of the recipient;
wherein the one or more potential transplant donors is identified as a transplant match and/or best transplant match for a recipient in need of a transplant if the gene dosage map of the one or more transplant donors correlates with the gene dosage map of the recipient, and
selecting tissue from a transplant donor having a gene dosage map that correlates with the gene dosage map of the recipient for transplant to the recipient.

In one embodiment, the method developed by the inventors disclosed herein may be used for identifying one or more potential transplant donors for a recipient in need of a transplant wherein the likelihood of developing graft versus host disease (GVHD) is reduced. In another embodiment, the method developed by the inventors disclosed herein may be used for identifying one or more potential transplant donors for a recipient in need of a transplant where the transplant is for any type of transplant where transplant phenotype is observed based on sequence copy number differences.

The method developed by the inventors disclosed herein may be used for reducing the likelihood of a transplant recipient developing graft versus host disease, the method comprising generating sequences of a gene complex from a nucleic acid sample obtained from the one or more potential transplant donors and the recipient, assigning a plurality of the generated sequences corresponding to each locus of the gene complex, determining gene dosage for each locus of the gene complex from the plurality of assigned sequences, generating a gene dosage map of the gene complex for the one or more potential transplant donors and the recipient from the determined gene dosage for each locus of the gene complex, and comparing the generated gene dosage map of the one or more potential transplant donors with the generated gene dosage map of the recipient, wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors.

The method developed by the inventors disclosed herein may be used for reducing the likelihood of a transplant recipient developing graft versus host disease (GVHD), the method comprising:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors.

The method developed by the inventors disclosed herein may be used for reducing the likelihood of a transplant recipient developing graft versus host disease (GVHD), the method comprising:
a) generating sequences of a gene complex from a nucleic acid sample from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors.

The method developed by the inventors disclosed herein may be used for reducing the likelihood of a transplant recipient developing graft versus host disease (GVHD), the method comprising:
a) generating sequences of a gene complex from a nucleic acid sample from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
   wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors, and selecting tissue from a transplant donor having a gene dosage map that correlates with the gene dosage map of the recipient for transplant to the recipient.

The method developed by the inventors disclosed herein may be used for reducing the likelihood of a transplant recipient developing graft versus host disease (GVHD), the method comprising:
a) generating sequences of HLA gene complex from a nucleic acid sample from the one or more potential transplant donors and the recipient;
b) assigning a plurality of the sequences generated in step (a) corresponding to each locus of the HLA gene complex;
c) determining gene dosage for each locus of the HLA gene complex from the plurality of sequences assigned in step (b);
d) generating a gene dosage map of the HLA gene complex for the one or more potential transplant donors and the recipient from the gene dosage for each locus of the HLA gene complex determined in step (c); and
e) comparing the generated HLA gene dosage map of the one or more potential transplant donors with the generated HLA gene dosage map of the recipient;
wherein the gene dosage map of the HLA gene complex the one or more potential transplant donors correlates with the gene dosage map of the HLA gene complex of the recipient in need of a transplant is indicative of reduced likelihood of the transplant recipient developing graft versus host disease following transplantation of a graft from the one or more transplant donors, and selecting tissue from a transplant donor having a gene dosage map of the HLA gene complex that correlates with the gene dosage map of the HLA gene complex of the recipient.

The methods disclosed herein may be used for transplanting tissue from one or more potential transplant donors to a recipient, comprising:

(i) identifying one or more potential transplant donors for a recipient in need of a transplant comprising the steps of:
a) generating sequences of a gene complex from a nucleic acid sample obtained from the one or more potential transplant donors and the recipient;
b) assigning a plurality of the sequences generated in step (a) corresponding to each locus of the gene complex;
c) determining gene dosage for each locus of the gene complex from the plurality of sequences assigned in step (b);
d) generating a gene dosage map of the gene complex for the one or more potential transplant donors and the recipient from the gene dosage for each locus of the gene complex determined in step (c); and
e) comparing the generated gene dosage map of the one or more potential transplant donors with the generated gene dosage map of the recipient;
wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors, and
(ii) transplanting tissue from a transplant donor having a gene dosage map that correlates with the gene dosage map of the recipient to the recipient.

The methods disclosed herein may be used for transplanting tissue from one or more potential transplant donors to a recipient, comprising:
(i) identifying one or more potential transplant donors for a recipient in need of a transplant comprising the steps of:
   a) generating sequences of a gene complex from a nucleic acid sample from the one or more potential transplant donors and the recipient;
   b) assigning a plurality of the sequences generated in step (a) corresponding to each locus of the gene complex;
   c) determining gene dosage for each locus of the gene complex from the plurality of sequences assigned in step (b);
   d) generating a gene dosage map of the gene complex for the one or more potential transplant donors and the recipient from the gene dosage for each locus of the gene complex determined in step (c); and
   e) comparing the generated gene dosage map of the one or more potential transplant donors with the generated gene dosage map of the recipient;
      wherein the gene dosage map of the one or more potential transplant donors correlates with the gene dosage map of the recipient in need of a transplant is indicative of reduced likelihood of the transplant recipient developing graft versus host disease following transplantation of a graft from the one or more transplant donors, and
(ii) transplanting tissue from a transplant donor having a gene dosage map that correlates with the gene dosage map of the recipient to the recipient.

The method developed by the inventors disclosed herein may be used for transplanting tissue from one or more potential transplant donors to a recipient, comprising:
(i) identifying one or more potential transplant donors for a recipient in need of a transplant comprising the steps of:
   a) generating sequences of HLA gene complex from a nucleic acid sample obtained from the one or more potential transplant donors and the recipient;
   b) assigning a plurality of the sequences generated in step (a) corresponding to each locus of the HLA gene complex;
   c) determining gene dosage for each locus of the HLA gene complex from the plurality of sequences assigned in step (b);
   d) generating a gene dosage map of the HLA gene complex for the one or more potential transplant donors and the recipient from the gene dosage for each locus of the HLA gene complex determined in step (c); and
   e) comparing the generated gene dosage map of the HLA gene complex of the one or more potential transplant donors with the generated gene dosage map of the HLA gene complex of the recipient;
      wherein the gene dosage map of the HLA gene complex of one or more potential transplant donors correlates with the gene dosage map of the HLA gene complex of the recipient in need of a transplant is indicative of reduced likelihood of the transplant recipient developing graft versus host disease following transplantation of a graft from the one or more transplant donors, and
(ii) transplanting tissue from a transplant donor having a gene dosage map of the HLA gene complex that correlates with the gene dosage map of the HLA gene complex of the recipient.

In one embodiment, the graft versus host disease (GVHD) disease may be acute graft-versus-host disease (aGVHD). In another embodiment, the graft versus host disease (GVHD) disease may be chronic graft-versus-host disease (cGVHD).

In one embodiment, the nucleic acid sample from the one or more donors and the recipient may be derived from tissues in the form of a tissue biopsy from the one or more donors and the recipient. The tissue biopsy may be biopsies from the skin, stomach, muscle or colon tissues from the one or more donors and the recipients. For a transplant recipient, the tissue may be a sample in the form of a tissue biopsy removed from an affected part of the human body of the transplant recipient. In one embodiment, for the one or more transplant donors, the tissue may be a sample in the form of a tissue biopsy removed from the same part of the human body as that obtained from the transplant recipient.

The method developed by the inventors disclosed herein may also be used for analysing sequences to identify one or more potential transplant donors for a recipient in need of a transplant, the method comprising generating sequences of a gene complex from a nucleic acid sample obtained from the one or more potential transplant donors and the recipient, assigning a plurality of the generated sequences corresponding to each locus of the gene complex, determining gene dosage for each locus of the gene complex from the plurality of assigned sequences, generating a gene dosage map of the gene complex for the one or more potential transplant donors and the recipient from the determined gene dosage for each locus of the gene complex, and comparing the generated gene dosage map of the one or more potential transplant donors with the generated gene dosage map of the recipient, wherein the one or more potential transplant donors is identified as a transplant match and/or best transplant match for a recipient in need of a transplant if the gene dosage map of the one or more transplant donors correlates with the gene dosage map of the recipient. In one embodiment, the transplant may be a graft and/or tissue and/or organ. In another embodiment,

The methods disclosed herein may comprise generating a gene dosage map for any gene complex or gene block. In one embodiment the gene complex or gene block is the HLA gene complex or HLA gene block or MHC gamma block or MR gene complex or Rhesus gene complex or any other gene complex relating to a transplant whose transplant phenotype is based on sequence copy number differences. The methods disclosed herein may comprise generating a gene dosage map for any gene complex or gene block pertaining to transplantation. In one embodiment, the gene complex or gene block pertaining to transplantation is the HLA gene complex or HLA gene block or any other gene complex relating to a transplant whose transplant phenotype is based on sequence copy number differences. The methods disclosed herein may comprise developing a gene dosage map for a highly polymorphic gene complex or a highly polymorphic gene block. In one embodiment the highly polymorphic gene complex or the highly polymorphic gene block is the HLA gene complex or HLA gene block or MHC gamma block or MR gene complex. The methods disclosed herein may comprise developing a gene dosage map for a polymorphic gene complex or a polymorphic gene block pertaining to transplantation. In one embodiment, the gene dosage map for a polymorphic gene complex or a polymorphic gene block pertaining to transplantation is the gene dosage map for the HLA gene complex or HLA gene block or MR gene complex or any other gene complex pertaining to a transplant whose transplant phenotype is based on sequence copy number differences. The methods disclosed herein may be used for a highly polymorphic gene complex or gene block where the gene complex or gene block is the HLA gene complex or MCH gamma block or MR gene complex or any other gene complex relating to a transplant whose transplant phenotype is based on sequence copy number differences.

The present disclosure provides a gene dosage map for each locus of a gene complex for one or more potential donors and a recipient generated using the methods disclosed herein.

The present disclosure provides a gene dosage map for each locus of HLA gene complex for one or more potential donors and a recipient generated using the methods disclosed herein.

The present disclosure provides a gene dosage map for each locus of MHC gamma block for one or more potential donors and a recipient generated using the methods disclosed herein.

The present disclosure provides use of a gene dosage map for each locus of a gene complex for one or more potential donors and a recipient generated using the methods disclosed herein for:
a) identifying one or more potential transplant donors for a recipient in need of a transplant;
b) reducing the likelihood of a transplant recipient developing graft versus host disease (GVHD);
c) treating graft versus host disease (GVHD) disease between one or more potential transplant donors and a recipient;
d) determining gene copy number difference; and
e) determining zygosity for each locus and all loci of the gene complex.

The present disclosure provides use of a gene dosage map for each locus of HLA gene complex for one or more potential donors and a recipient generated using the methods disclosed herein for:
a) identifying one or more potential transplant donors for a recipient in need of a transplant;
b) reducing the likelihood of a transplant recipient developing graft versus host disease (GVHD);
c) treating graft versus host disease (GVHD) disease between one or more potential transplant donors and a recipient;
d) determining gene copy number differences; and
e) determining zygosity for each locus and all loci of HLA gene complex.

Using the methods disclosed herein, in one embodiment, gene copy number difference may be 0 or may be 1 or may be 2 or may be 3 or may be more than 3.

### Preparation of Nucleic Acid

The method of the invention may be performed on a nucleic acid sample that has already been obtained prior, or obtained freshly, from a subject using any suitable technique known in the art. As disclosed herein, the method may comprise obtaining the nucleic acid sample from the one or more transplant donors and the recipient in need of a transplant is genomic DNA extracted from a biological sample. As used herein, a “biological sample” may be for instance lymphocytes, whole blood, buccal swab, biopsy sample or frozen tissue or any other sample comprising genomic DNA. The whole blood may be anti-coagulated whole blood. It is also possible to utilize samples obtained through non-invasive means, for example by way of cheek swab or saliva-based DNA collection. Various suitable methods for extracting DNA from such sources are known in the art. These range from organic solvent extraction to absorption onto silica-coated beads and anion exchange columns. Automated systems for DNA extraction are also available commercial and may provide good quality, high purity DNA. The nucleic acid used in the method of the invention may be single-stranded and/or double stranded genomic DNA. In the method disclosed herein, the genomic DNA may be at concentration of about 10 ng/μl to about 100 ng/μl.

In some embodiments, the nucleic acid may include long nucleic acids comprising a length of at least about 1 kb, at least about 2 kb, at least about 5 kb, at least about 10 kb, or at least about 20 kb or longer. Long nucleic acids can be prepared from sources by a variety of methods well known in the art. Methods for obtaining biological samples and subsequent nucleic acid isolation from such samples that maintain the integrity (i.e. minimize the breakage or shearing of nucleic acid molecules are preferred. Exemplary methods include, but are not limited to, lysis methods without further purification (e.g. chemical or enzymatic lysis method using detergents, organic solvents, alkaline, and/or proteases), nuclei isolation with or without further nucleic acid purification, isolation methods using precipitation steps, nucleic acid isolation methods using solid matrices (e.g. silica based membranes, heads, or modified surfaces that bind nucleic acid molecules), gel-like matrices (e.g. agarose) or viscous solutions, and methods that enrich nucleic acid molecules with a density gradient.

In one embodiment, the nucleic acids used in the method of the invention are fragmented in order to obtain a desired average fragment size. In one embodiment, the method as disclosed herein may comprise the nucleic acid sample is fragmented before being contacted with the one or more oligonucleotide probes. In another embodiment, the method as disclosed herein may comprise the nucleic acid sample is fragmented after being contacted with the one or more oligonucleotide probes. In one embodiment, the oligonucleotide probe may be a DNA-based probe. In another embodiment, the oligonucleotide probe may be a RNA-based probe.

The skilled person will appreciate that the required length of nucleic acid fragment will depend on the sequencing technology that is used. For example, the Ion Torrent utilise fragments from around 100 bp to 200 bp in length whereas the Pacific Biosciences NGS platform can utilise nucleic acids fragments up to 20 kb in length.

The nucleic acid may be fragmented by physical shearing, sonication, restriction digestion, or other suitable technique known in the art. The fragmenting of the nucleic acid can be performed so as to generate nucleic acid fragments having a desired average length for use in the preparation of a DNA library. As disclosed herein, the method may comprise the fragments of the nucleic acid sample have an average length greater than about 100 bp. For example, the length or the average length, of the nucleic acid fragments may be at least about 100 bp, at least about 200 hp, at least about 300 bp, at least about 400 bp, at least about 500 bp, at least about 600 bp, at least about 700 bp, at least about 800 bp, at least about 900 bp, at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 15 kb or at least about 20 kb.

### Preparation of DNA Library

A DNA library is prepared using the extracted nucleic acid. The nucleic acid may be genomic DNA. The DNA library may be prepared using any commercially available kit that adds adapter sequences onto the ends of DNA fragments to generate indexed libraries for single-read or paired-end sequencing. The DNA library of the present disclosure was prepared using the commercially available Nextera Flex for enrichment kit by Illumina as per manufacturer's instructions. In one embodiment, a library for a 550 bp insert size may be prepared for which 100 ng genomic DNA may be used. In another embodiment, a library for 350 bp insert size may be prepared for which 100 ng genomic DNA may be used. The library may be a fragmented shotgun library.

In one embodiment, the nucleic acid sample may be treated in order to generate single-stranded nucleic acid, or to generate nucleic acid comprising a single-stranded region, prior to contacting the sample with oligonucleotide probes. The nucleic acid can be made single stranded using techniques known in the art, for example, including known hybridization techniques and commercially available kits such as the ReadyAmp™ Genomic DNA Purification System (Promega). Alternatively, single stranded regions may be introduced into a nucleic acid using a suitable nickase in conjunction with an exonuclease. The methods disclosed herein may comprise the nucleic acid sample from the one or more transplant donors and the recipient in need of a transplant that is contacted with the one or more oligonucleotide probes being a single stranded nucleic acid.

The fragmented shotgun library is subjected to hybridization with DNA oligonucleotides or “probes”. The term “probe” or “oligonucleotide probe” according to the present invention refers to an oligonucleotide which is designed to specifically hybridize to a nucleic acid of interest where the nucleic acid of interest is a locus of the HLA gene complex. Preferably, the probes are suitable for use in preparing nucleic acid for NGS sequencing using a hybrid-capture technique.

As used herein, the term “hybrid-capture technique” refers to a target-enrichment strategy using hybrid capture where the technique works by capturing adaptor-modified genomic DNA of interest by hybridization to target-specific probes either on a microarray surface or in solution, which are then isolated by magnetic pulldown. This technique may be used for analyzing specific genetic variants in a given sample. In the present disclosure, the hybrid-capture technique may be used to capture all alleles of every gene loci of the HLA complex.

In one embodiment, the probe may be stable for target capture and be around 60 to 120 nucleotides in length. Alternatively, the probe may be about 10 to 25 nucleotides. In certain embodiments, the length of the probe is 10, 11, 12, 13, 14, 15, 16, 17, 18, 19, 20, 21, 22, 23, 24 or 25 nucleotides. The oligonucleotide probes as used in the present invention may be ribonucleotides, deoxyribonucleotides and modified nucleotides such as inosine or nucleotides containing modified groups which do not essentially after their hybridization characteristics

There may be multiple different probes which specifically hybridize to multiple different loci of the HLA gene complex. The probes of the present disclosure may capture alleles of the loci of the HLA complex with a sequence difference from about 1% to about 20%. For example, the probes may capture alleles with sequence difference in the range of about 1% to about 20%, such as about 3% to about 18%, such as about 5% to about 15%, such as about 8% to 15% and such as about 10% to about 12%.

Compared to PCR-based amplicon sequencing, hybridization-based enrichment sequencing can target a higher amount of total gene content and support more comprehensive profiling of all variant types. The larger amount of total gene content allows for the characterization of both known and novel variants for discovery-related applications.

As used herein, the term “hybridization” refers to the process in which an oligonucleotide probe binds non-covalently with a target nucleic acid to form a stable double-stranded polynucleotide. Hybridization conditions will typically include salt concentrations of less than about 1 M, more usually less than about 500 mM and may be less than about 200 mM. A hybridization buffer includes a buffered salt solution such as 5% SSPE, or other such buffers known in the art. Hybridization temperatures can be as low as 5° C. but are typically greater than 2° C. and more typically greater than about 30° C., and typically in excess of 37° C. Hybridizations are usually performed under stringent conditions, i.e. conditions under which a probe will hybridize to its target sequence to which it is complementary, but will not hybridize to the other, non-complementary sequences. As used herein the term “complementary” and grammatical equivalents refer to the nucleotide base-pairing interaction of one nucleic acid with another nucleic acid, including modified nucleic acids and. nucleic acid analogues, that results in the formation of a duplex triplex, or other higher-ordered structure. The primary interaction is typically nucleotide base specific, e.g. A:T, A:U, and G:C, by Watson-Crick and Hoogsteen-type hydrogen bonding. Conditions under which oligonucleotide probes anneal to complementary or substantially complementary regions of target nucleic acids well known in the art. In one embodiment, hybridization may be performed using array-based hybrid capture method. In another embodiment, hybridization may be performed using in-solution hybrid capture method.

In one embodiment, the one or more oligonucleotide probes used in the method of the present invention comprises a capture tag to facilitate enrichment of nucleic acid of interest bound to an oligonucleotide probe from other nucleic acid sequences in a sample. In one embodiment, hybridization-based enrichment strategy for next generation sequencing may be used. In order to enrich for the nuclei acid of interest from other nucleic acid sequences, the capture tag binds to a suitable binding agent. As would be understood in the art, the phrase “enriching for a nucleic acid” refers to increasing the amount of a target nucleic acid sequence in a sample relative to nucleic acid that is not bound to an oligonucleotide probe. Thereby, the ratio of target sequence relative to the corresponding non-target nucleic acid in a sample is increased. In one embodiment, the capture tag is a “hybridization tag”. As used herein, the term “hybridization tag” and grammatical equivalents can refer to a nucleic acid comprising a sequence complementary to at least a portion of another nucleic acid sequence that acts as the binding agent (i.e. a “binding tag”). The method disclosed herein may further comprise contacting the capture tag with a binding agent. In one embodiment, the capture tag may be biotin or streptavidin. The degree of complementarity between a hybridization tag and a corresponding binding tag sequence can vary with the application, in some embodiments, the hybridization tag can be complementary or substantially complementary to a binding tag or portions thereof. For example, a hybridization tag can comprise a sequence having a complementarity to a corresponding binding tag of at least about 50%, at least about 60%, at least about 70%, at least about 80%, at least about 90% and at least about 99%. In some embodiments, a hybridisation tag can comprise a sequence having 100% complementarity to a corresponding biding tag. In some embodiments, a capture probe can Include a plurality of hybridization tags for which the corresponding binding tags are located in the same nucleic acid, or different nucleic acids. In certain embodiments, a hybridization tag can comprise at least about 5 nucleotides, at least about 10 nucleotides, at last about 15 nucleotides, at least about 20 nucleotides, at least about 5 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, and at least about 100 nucleotides.

In another embodiment, the capture tag may comprise an “affinity tag”. As used herein, the term “affinity tag” can refer to a component of a multi-component complex, wherein the components of the multi-component complex specifically interact with or bind to each other. For example, an affinity tag can include biotin that can bind streptavidin. Other examples of multiple-component affinity tag complexes include, ligands and their receptors, for example avidin-biotin, streptavidin-biotin, and derivatives of biotin, streptavidin, or avidin.

Thus, the binding agent used in the method of the invention is capable of binding to an affinity tag as described herein to facilitate separation of a nucleic acid of interest from other nucleic acid sequences in a sample. For example, in one embodiment, the affinity tag comprises biotin and the binding agent comprises streptavidin. In another embodiment, the binding agent may be biotin or streptavidin. The binding agent is typically on a substrate. Examples of substrates include beads, microspheres planar surfaces, columns, wells and the like. The terms “microsphere” or “bead” or “particle” or grammatical equivalents are understood in the art and refer to a small discrete particle. The composition of the substrate will vary on the application. Suitable compositions include those used in peptide, nucleic acid and organic moiety synthesis, including, but not limited to plastics, ceramics, glass or any other suitable material. The beads may be in any shape or form as long as the beads are able to perform its function. The beads may be spherical, near spherical or irregular in shape. The size of the beads used may range in sizes from about 100 nm to about 1 mm depending on the need. In some embodiments, a substrate can comprise a metallic composition, for example, ferrous, and may also comprise magnetic properties. In one embodiment, the substrate may be a magnetic substrate. In one embodiment, the substrate may be a magnetic bead. For example, in one embodiment, utilizing magnetic beads may include capture probes comprising streptavidin-coated magnetic beads. In addition, the beads may be porous, thus increasing the surface area of the bead available for association with capture probes. The bead sizes range from nanometers, for example, 100 nm, to millimeters, for example, 1 mm, with beads from about 0.2 μm to about 200 μm, or from about 0.5 to about 5 μm, although in some embodiments smaller beads may be used. The binding agent may be coated on or attached to a suitable substrate such as, for example, a microsphere or bead. In some embodiments, the substrate may be magnetic to facilitate enrichment of a target nucleic acid of interest. In one embodiment, hybridization-based enrichment strategy for next generation sequencing may be performed on a microarray surface. In one embodiment, hybridization-based enrichment strategy for next generation sequencing may be performed in solution.

In other embodiments of the present invention, other target enrichment strategies may be used in next generation sequencing (NGS) workflows to eliminate genomic DNA regions that are not of interest for a particular experiment such as, for example, transposon-mediated fragmentation (tagmentation), molecular inversion probes (MIPs), and singleplex and multiplex polymerase chain reaction (PCR) target enrichment.

### Hybrid-Capture Next Generation (NGS) Sequencing

Sequencing was conducted directly after nucleic acid extraction and library preparation. The sequencing may be high-throughput sequencing. According to the methods disclosed herein, the high-throughput sequencing may be hybrid-capture next generation sequencing (NGS). Hybrid-capture NGS sequencing may be conducted using any commercially available compatible sequencing kit and any suitable commercially available sequencing platform. During sequencing, specific motifs, all exons, or a whole gene may be sequenced.

The method disclosed herein may comprise sequencing of a gene and/or gene complex and/or gene block, where the gene may be a highly polymorphic gene, the gene complex may be a highly polymorphic gene complex and the gene block may be a highly polymorphic gene block. The gene may be a gene pertaining to transplantation. The gene may be a highly polymorphic gene pertaining to transplantation. In one embodiment, the gene is a HLA gene. The gene complex may be a gene complex pertaining to transplantation. The gene complex may be a highly polymorphic gene complex pertaining to transplantation. In one embodiment, the highly polymorphic gene complex may be a HLA gene complex. In another embodiment, the highly polymorphic gene complex may be a MHC gene complex. The gene block may be a gene block pertaining to transplantation. The gene block may be a highly polymorphic gene block pertaining to transplantation. In one embodiment, the highly polymorphic gene block may be the MHC gamma block. In one embodiment, the gene complex may the HLA gene complex or the MHC gamma block or MR gene complex or Rhesus gene complex or any gene complex relating to a transplant whose transplant phenotype is based on sequence and/or gene copy number differences.

The method disclosed herein comprises a method for generating sequences of a gene complex from a nucleic acid sample obtained from the one or more potential transplant donors and the recipient, is a method for identifying gene alleles in the one or more transplant donors and the recipient in need of a transplant, the method comprising: contacting a nucleic acid sample from the one or more transplant donors and the recipient with oligonucleotide probes, wherein the oligonucleotide probes hybridize to gene target sequences in the nucleic acid sample; enriching a nucleic acid by hybridizing the nucleic acid to one or more oligonucleotide probes; separating nucleic acid hybridized to the one or more oligonucleotide probes from nucleic acid not hybridized to the one or more oligonucleotide probes; and sequencing the enriched nucleic acid to identify one or more gene alleles; wherein the gene target sequences are in a non-coding region of the gene.

As disclosed herein, the method may comprise amplifying the nucleic acid bound to the one or more oligonucleotide probes. The method disclosed herein may comprise sequencing an HLA gene exon or any gene exon pertaining to transplantation. The method disclosed herein may comprise sequencing of the entire HLA gene or an entire gene pertaining to transplantation.

In the present disclosure, whole sequence reads of every loci of the HLA gene complex may be sequenced. In the present disclosure, NGS was conducted using the MiSeq, iSeq, or MiniSeq using Illumina 2×300 bp sequencing protocol. Sequencing reads are produced in the form of deconvoluted (de-indexed) patient-specific sequence reads. Platforms for next-generation sequencing using the method disclosed herein may include any suitable platform that is commercially available, but are not limited to: Illumina's MiSeq, iSeq, or MiniSeq Systems. In one embodiment, the sequences are gene sequences. In another embodiment, the sequences are intergenic sequences. In another embodiment, the sequences are gene sequences and intergenic sequences.

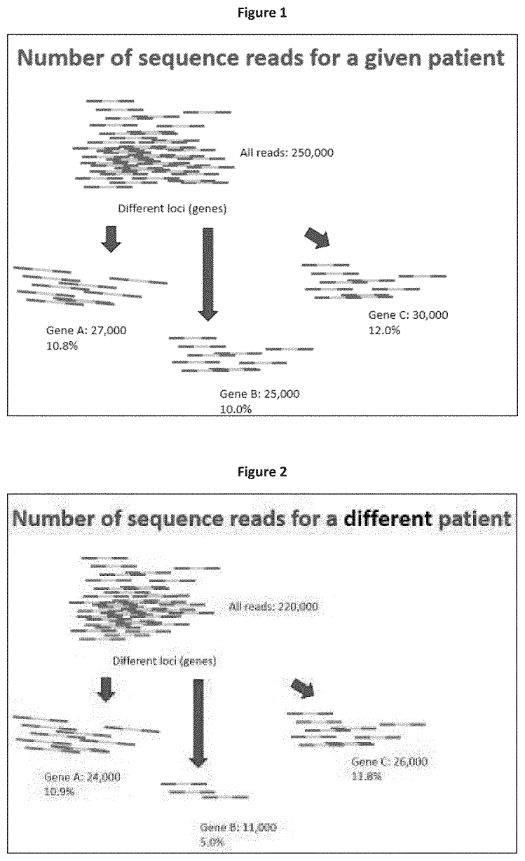

The method disclosed herein may comprise the sequences being generated in a computer readable form. In one embodiment, the computer readable form may be FASTQ. In another embodiment, the computer readable form may be FASTA. In yet another embodiment, the computer readable form may be GZ. FIGS. 1 and 2 exemplify the total number of NGS reads that may be generated for all loci of the HLA gene complex which may be next assigned into gene-specific allocations using a sequence program to analyse, edit and align the generated NGS sequences. NGS sequence reads that are poor in quality with high background noise or low depth of sequencing coverage are not assigned by the software into gene specific allocations, and are termed as “unassigned reads”.

The present hybrid-capture NGS technique using probes is suited to the identification of alleles in highly polymorphic genes. As used herein, the term “highly polymorphic gene” includes reference to genes that have greater levels of polymorphism in the coding region of the gene compared to the non-coding regions. For example, a highly polymorphic gene may have a greater number of polymorphisms per kb of coding sequence when compared to the number of polymorphisms per kb of non-coding sequence of the gene. Well known examples of highly polymorphic genes are the human leukocyte antigen (HLA) genes, which is the human version of the MHC complex. The coding regions of HLA molecules are highly polymorphic as it is thought they are under positive select pressure to evolve in response to pathogenic threat. The non-coding regions of HLA are not under such selective pressure and do not share the same degree of polymorphism. While the non-coding regions of HLA class I are polymorphic, the polymorphisms are not randomly distributed across these regions and closely related, by coding sequence similarity, have identical non-coding sequences. The hybrid-capture NGS technique uses probes designed explicitly to the non-coding regions of HLA.

### Assignment of NGS Sequences

From the total number of sequences generated using NGS based on amplification of DNA material from the fragment shotgun library, these sequences may be allocated into gene-specific allocations using a suitable proprietary software program or any other suitable software program that is commercially available. To accurately allocate or assign the NGS sequences into gene-specific allocations, the software program may be used to analyse, edit and align the generated NGS sequences in comparison against a known library of HLA alleles. In the present disclosure, a plurality of the sequences generated using NGS may be assigned using a computer program. The computer program may be a sequence editing and alignment program. In one embodiment, the sequence editing and alignment program is the Assign™ TruSight version 2.1 software (“Assign” software) by CareDx Inc. In another embodiment, the sequence editing and alignment software program is the AlloSeq Assign software by CareDx. The sequence editing and alignment program may be Assign™ TruSight version 2.1 software and/or AlloSeq Assign software.

In the present disclosure, the software program that may be used to analyse, edit and align the generated NGS sequences to the reference library of known HLA alleles is the Assign™ TruSight version 2.1 proprietary software by CareDx Inc and/or AlloSeq Assign software by CareDx Inc.

The library of known HLA alleles may be the IMGT/HLA library which is a specialist database that comprises all known sequences human major histocompatibility complex, known as the human leukocyte antigen (HLA). The IMGT/HLA database includes sequences for the World Health Organization (WHO) Nomenclature Committee for Factors of the HLA System. The IMGT/HLA database is part of the international ImMunoGeneTics (IMGT) project (www.imgt.org).

The Assign software and/or AlloSeq Assign software assists with the assignment of a human leukocyte antigen (HLA) type. The software is designed to analyse data from libraries prepared with the CareDx AlloSeq Sequencing Panels and then sequenced on an Illumina sequencer. The Assign software was used to import the NGS sequence data, perform base calling, edit sequences which results in edited sample sequences which are then compared to known sequences contained in the IMGT/HLA database of alleles.

A first step in using the Assign software and/or AlloSeq Assign software is to import the generated NGS sequence reads. The Assign software may be used to analyse the imported sequences. Analysis may include alignment of reads, base calling, phasing, IMGT/HLA reference alignment, and HLA typing.

The second step, is to analyse, annotate and allocate the imported NGS reads into gene specific allocations. The NGS reads were compared against a library of known HLA alleles which have been categorised in accordance with the nomenclature of HLA alleles. The library of known HLA alleles may be a library of known HLA allele motifs. Each HLA allele name has a unique number corresponding to up to four sets of digits separated by colons. The length of the allele designation is dependent on the sequence of the allele and that of its nearest relative. All alleles receive at least a two digit name, which corresponds to the first two digits, the digits before the first colon describe the type, which often corresponds to the serological antigen carried by an allotype. The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns, or in the 5′ or 3′ untranslated regions that flank the exons and introns, are distinguished by the use of the fourth set of digits.

To explain the HLA nomenclature, the example HLA-A*02:01:01:02L, is used with reference to the following table below.

| | |
|---|---|
| HLA | The HLA Prefix |
| - | The hyphen separates the gene name from the HLA prefix. |
| A | The gene name. |
| | For TruSight HLA, the gene name can be A, B, C, DRB1, |
| | DRB3, DRB4, DRB5, DQB1, DPB1, DQA1, or DPA1. |
| * | The asterisk separates the gene name from the sequence |
| | information. |
| 02 | Field 1—The allele group; alleles that encode an antigen. |
| : | A colon separates fields. |
| 01 | Field 2—Specific alleles that differ at the protein level from |
| | DNA substitutions and result in nonsynonymous amino acid |
| | substitutions. |
| : | A colon separates fields. |
| 01 | Field 3—Synonymous DNA substitutions within coding |
| | regions of the gene. |
| : | A colon separates fields. |
| 02 | Field 4—Differences in the noncoding regions of the gene. |
| L | This expression modifier is present regardless of the number |
| | of fields reported. As of date, the following modifiers are |
| | possible: |
| | N denotes Null—An allele that is not expressed. |
| | L denotes Low—An allele encoding a protein with significantly |
| | reduced or low cell surface expression. |
| | S denotes Secreted—An allele encoding a protein that is |
| | expressed as a secreted molecule only. |
| | Q denotes Questionable—An allele with a mutation that has |
| | previously been shown to have a significant effect on cell |
| | surface expression, but is not confirmed. Therefore, its |
| | expression remains questionable. |

Any NGS sequence reads that have a similar sequence in comparison to any of the sequences recorded in the IMGT/HLA allele library will be automatically assigned into gene specific allocations. Any NGS sequence reads that are unreadable, with high background noise and/or have high base mismatches will not be assigned into gene specific allocations and are termed as “unassigned reads”.

The term “G Group” as used herein refers to G codes for reporting of ambiguous allele typings, which are HLA alleles that have identical nucleotide sequences across the exons encoding the peptide binding domains (exon 2 and 3 for HLA class I and exon 2 only for HLA class II alleles), will be designated by an upper case ‘G’ which follows the first 3 fields of the allele designation of the lowest numbered allele in the group. The group designation will contain a minimum of six digits.

The term “P Group” as used herein refers to P codes for reporting of ambiguous allele typings, which are HLA Sequences having the same antigen binding domains. This analysis is performed on the protein sequence, and for HLA Class I alleles, identity in the ‘antigen binding domains’ is based on identical protein sequences as encoded by exons 2 and 3. For HLA Class II alleles this is based on identical protein sequences as encoded by exon 2. HLA alleles having nucleotide sequences that encode the same protein sequence for the peptide binding domains (exon 2 and 3 for HLA class I and exon 2 only for HLA class II alleles) will be designated by an upper case ‘P’ which follows the 2 field allele designation of the lowest numbered allele in the group. The group designation will contain a minimum of four digits.

As used herein, the term “base calling” refers to the process of assigning bases using the Assign software for a sample of the one or more donors and/or a sample of a recipient at a given reference nucleotide position.

The methods disclosed herein may comprise assigning a plurality of the sequences generated from hybrid-capture NGS sequencing corresponding to each locus of the gene complex based on: one or more regions of each locus; all exons in each of the locus; and/or an entire sequence for each locus.

After analysis of the imported files, the consensus sequence of the analysed files may be aligned with reference sequences, the reference sequences being the library of known HLA alleles from the IMGT/HLA library. Sample consensus sequence are compared to a panel which lists all the IMGT/HLA allele pairs that exactly match or closely match the sample consensus sequence (refer to FIG. 18). Doing this provides information for each of the allele pairs listed and whether there are any mismatches in the allele pairs. Allele pairs with no mismatches appear at the top of the columns followed by pairs with increasing numbers of mismatches. When no heterozygous positions are detected in the sequence used for the typing (default is all exons), the Allele 2 column contains an X. The presence of an X does not constitute confirmation of homozygosity. When a heterozygous position is found in the active sequence, a second allele is reported. The allele pairs are banded white and gray by alternating rows for ease of viewing. Sometimes, the allele includes orange, which indicates that a part of the reference sequence is missing in the IMGT/HLA reference for that allele.

### Generating Gene Dosage and Gene Dosage Map

After assigning NGS sequences into the gene-specific allocations, every gene locus and/or all gene loci will have a plurality of reads unique to a subject as exemplified in FIGS. 1 and 2.

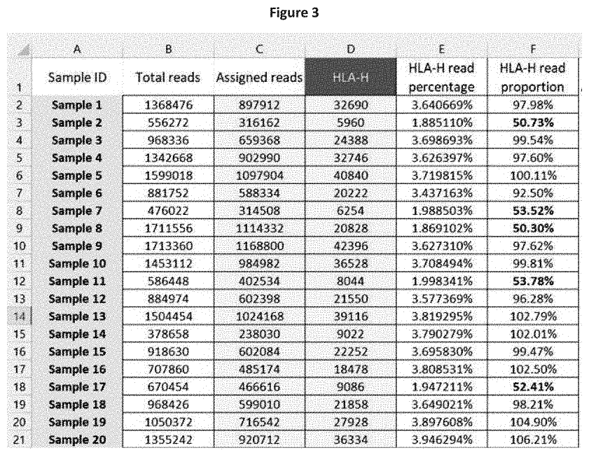

In order to compare the amount of sequence reads for a patient sample, at a given locus or loci, it is crucial that compared reads at a given locus are relative to total assigned sequence reads for all loci of the gene complex as exemplified in FIG. 3. According to the method disclosed herein, the gene dosage map for each locus of the gene complex for the one or more potential donors and the recipient may comprise dividing the plurality of sequences assigned to each locus by the plurality of sequences assigned to all loci of the gene complex. In FIG. 3, the amount of sequence reads of a subject for the HLA-H gene for example, is obtained by dividing the number of assigned reads in column D by the number of total assigned reads for all loci in column C, to produce a determined value of the HLA-H gene as a ratio of the mean proportion in column E which may then calculated as a percentage proportion in column F.

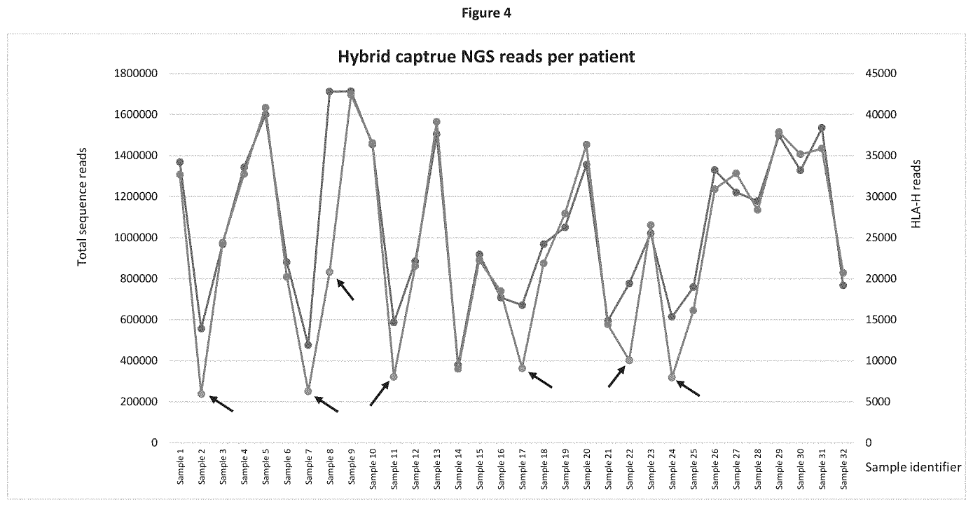

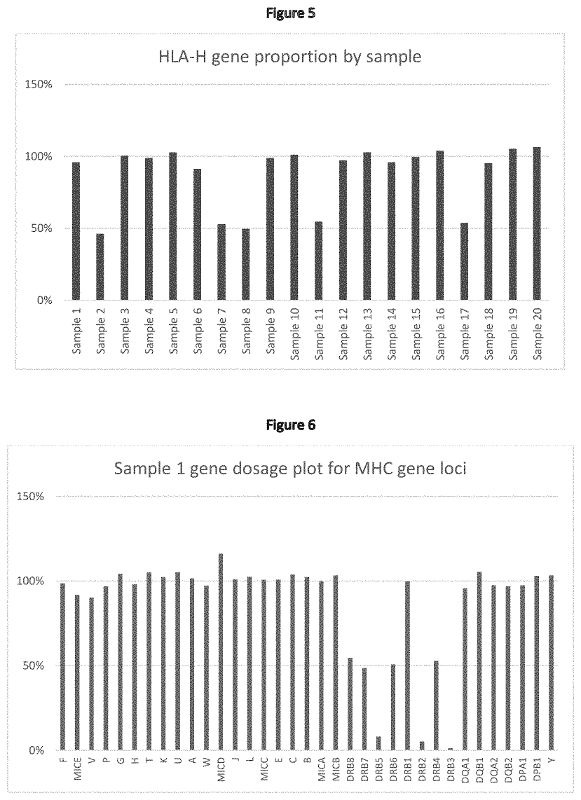

As shown in FIG. 4, several individuals (patients 2, 4, 7, 8, 11, 17, 22 and 24) denoted by arrows, are observed to have a reduction in sequence reads for the HLA-H locus compared to total sequence reads and this difference may be more overtly demonstrated via a ratio of the means proportion presented as percentage proportion (refer to FIGS. 3 and 5).

Gene dosage for a particular gene is obtained by dividing the number of assigned reads specific to a locus, for example, the HLA-H gene, by the total number of assigned sequence reads assigned to all loci for a gene complex, the method as disclosed herein provides the advantage of a locus-specific proportion of reads for a subject. The method disclosed herein also provides the advantage of being able to determine the copy number of each locus and all loci of the gene complex to allow determination of zygosity for each locus and all loci of the gene complex. Most eukaryotes have two matching sets of chromosomes; that is, they are diploid. Diploid organisms have the same loci on each of their two sets of homologous chromosomes except that the sequences at these loci may differ between the two chromosomes in a matching pair and that a few chromosomes may be mismatched as part of a chromosomal sex-determination system. If both alleles of a diploid organism are the same, the organism is homozygous at that locus. If the alleles are of different nucleotide sequence make-up, the organism is heterozygous at that locus. If one allele is missing, an organism is termed a hemizygous, and, if both alleles are missing, it is nullizygous. Using the methods disclosed herein, the calculated copy number for each locus being presented as a percentage proportion (as exemplified in FIG. 3) will inform us if an individual is a homozygous, hemizygous or nullizygous. The same calculation process may be employed to obtain the gene dosage map of all or nearly all gene loci of a gene complex. The same calculation process may be employed to obtain the copy number for any gene complex relating to a transplant whose transplant phenotype can be observed based on sequence and/or gene copy number differences. The gene dosage map of all gene loci in a gene complex is collated to form the gene dosage map. The gene dosage map comprises the gene dosage for all or nearly all gene loci of a gene complex. In one embodiment, the gene dosage map for the HLA gene complex contains gene dosage for all or nearly all gene loci. The same calculation process may also be employed to obtain the copy number in sequences. Copy number measured may be 0, or may be 1, or may be 2 or may be 3 or may be more than 3. The same calculation process may also be employed to obtain the copy number for any event caused by chromosome recombination.

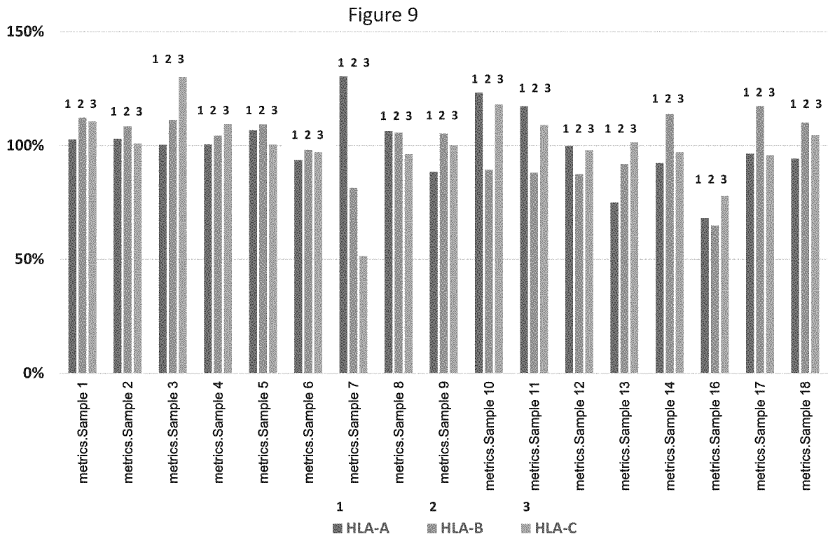

Referring to FIG. 4, determination of zygosity using the methods disclosed herein can be observed where patients 2, 7, 8, 11, 17, 22 and 24 (denoted by arrows) are hemizygous for the HLA-H gene, as these patients all possess only one copy of the HLA-H gene, from the percentage proportion of the number of HLA-H specific reads compared to the number of total assigned reads for all loci being approximately 50%. Such an explicit demonstration of difference in copy number leading to determination of zygosity in an individual or in multiple different individuals using the method disclosed herein would not have been demonstrated definitively via nucleotide sequencing (refer to FIG. 9). Commercial transplant matching methodologies are currently primarily PCR-based. Derivation of sequence dosage based on copy number directly from genomic DNA is not readily achievable via current PCR methodologies, where exponential propagation of DNA in single-plex through the multiple PCR cycles results in decreased uniformity between loci and patient samples which can be seen from FIG. 9.

Currently, PCR-based methods are widely used for gene copy number interpretation. These PCR-based methods specifically target regions of a sequence to exponentially increase DNA content, via successive cycling or thermal conditions. During the PCR cycle, PCR progresses through an exponential, or log phase until the reagents present within the reaction mixture begin to deplete. Depletion of PCR reagents within the reaction mixture causes the PCR reaction to reach a plateau phase, or lag phase. As such, the final yield i.e. DNA product is determined by reagent availability. In the majority of instances polymerase chain reactions proceed to the endpoint, whereby one limiting factor (dNTP, oligonucleotide primer, or other reagent) is depleted, given that the focus is on total DNA yield for downstream applications. When multiplexing PCR for products of varying length and G-C content, it is very difficult to ensure that the efficiency of each reaction within the PCR is directly comparable. Given that amplicons often reach the PCR endpoint the ability to compare gene dose based on copy number is greatly diminished via this method. While it may be possible to demonstrate dosage differences with PCR, this is more readily achieved via quantitative real time PCR (qPCR) where fewer cycles are employed and samples are compared for change in their cycle threshold, or signal, at a given number of PCR cycles relative to other samples and known input concentrations. Generation of enough amplicon to obtain adequate depth of sequence coverage, whilst also ensuring few enough cycles such that all reactions remain in the log phase of PCR, and using normalised starting input template DNA, means that PCR-based next generation sequencing results are often sub-optimal. In contrast, hybrid capture DNA sequence enrichment used in the methods disclosed herein uses few PCR cycles and coupled with the method disclosed herein to generate gene dosage for a particular gene, the obtained sequence reads are relative to starting material and copy number.

The methods disclosed herein allows capture and allows comparison of like concentrations of starting DNA and adjustment for total sequence reads (input DNA).

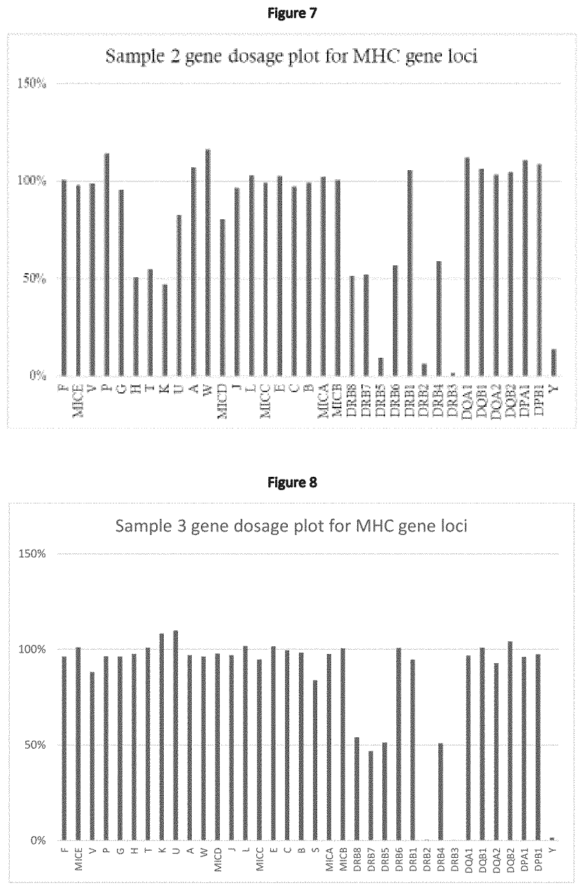

The method disclosed herein may comprise the gene dosage for each locus which is the copy number for each locus of the gene complex. Further examples for the demonstration of differing zygosity of gene loci in the HLA gene complex for multiple different patients is shown in FIG. 5. Demonstration of zygosity at a particular locus for three individuals or patients is shown in FIGS. 6 to 8.

Zygosity has immense relevance to transplant matching and standard methods do not readily differentiate homozygous (two copies of a gene (one per chromosome) from hemizygous (one copy on chromosome only, the other deleted) sequence. As such allele sequencing reports for transplant matching typically assume the presence of a second identical allele, with a disclaimer. Enumeration of gene copy number will allow definitive reporting of two alleles with identical sequence.

This may have further application where monitoring leukemoid changes in patients, whereby loss of heterozygosity (LOH) may have negative implications for patient survival. Demonstration of gene dose in the presence of LOH, distinguishes results from allele drop-out which may be observed using conventional PCR NGS methods. Similarly, re-emergence of recipient MHC sequence reads may be detected via changes in sequencer read count. Pseudogenes, non-specific gene targets, and expressed genes within the major histocompatibility complex (MHC) may vary by copy number (gene dose) across individuals.

Comparison of normalized sequence reads using the method disclosed herein facilitates the determination of zygosity for each locus, and differentiation of homozygous from hemizygous or null sequence. The methods disclosed herein provides a comparison of gene content/copy number profiles to allow better allele matching between donors and their recipients and better surveillance for patient who are post-transplant. Using the methods disclosed herein and the same calculation process disclosed herein, comparison of sequence and/or gene content/copy number profiles using the methods disclosed herein may be applied to reducing the likelihood or preventing graft versus host disease (GVHD) disease between one or more potential transplant donors and a transplant recipient. Using the methods disclosed herein and same calculation process disclosed herein, comparison of gene content/copy number profiles using the methods disclosed herein may also therefore be applied for reducing the likelihood or preventing any transplant rejection where transplant phenotype is observed based on gene content/copy number profiles and/or sequence copy number differences.

Besides determining zygosity, the methods disclosed herein may also comprise the determination of whether two alleles have an identical sequence. Two alleles for a gene may be compared using the Assign software. An individual may be termed ‘homozygous’ or a particular gene when identical alleles of the gene are present on both homologous chromosomes. An individual may be termed a ‘heterozygous’ at a gene locus when there are two different alleles of a gene.

By repeating the locus-specific analysis for all contiguous loci, for a given patient, the method disclosed herein may generate a gene dosage map for all loci across the HLA gene complex for that particular patient. The methods disclosed herein may be used to generate gene dosage maps of one or more transplant donors and a recipient in need of a transplant which will provide improved information on transplant matching. The methods disclosed herein comprises the gene dosage map being the copy number for all loci of the gene complex. The methods disclosed herein may comprise generating a gene dosage map for any other gene blocks or gene complexes. The methods disclosed herein may be used to generate a gene dosage map for any other highly polymorphic gene blocks or any other highly polymorphic gene complexes. A gene dosage map generated using the methods disclosed herein may comprise the copy number of each locus and all loci of the gene complex to allow determination of whether two alleles have an identical sequence. Using the methods disclosed herein, a gene dosage map may be generated for the HLA gene complex or MHC gamma block. Using the methods disclosed herein, a gene dosage map may be generated for any other gene complexes such as MR gene complex and Rhesus gene complex. Using the methods disclosed herein, a gene dosage map may be generated for any gene complex relating to transplant whose transplant phenotype is based on sequence and/or gene copy number differences.

Using the methods disclosed herein, the gene dosage map may produce a signature that indicates sequence similarities and differences between patients and donors. These sequence differences indicate haplotype differences and result in higher risk of poor transplant outcomes. The approach of comparing normalised sequence read count, across gene loci using the method disclosed herein provides a novel means of comparing gene content, in addition to but distinct from standard nucleotide sequence allele assignment methods. The ability to compare gene content/dosage has the advantageous potential to better match patients and donors across blocks of sequence that are not routinely investigated. Comparing multiple loci in a patient may advantageously allow for a patient-specific map of the MHC, which may be employed to better match a transplant recipient with their one or more potential donors.

The terms “patients”, “subjects” and “individuals” may be used interchangeably in the present disclosure but they refer to the one or more transplant donors that are being determined by the methods disclosed herein to be a good transplant match for the recipient in need of a transplant.

### Kits

The present disclosure provides a kit for identifying one or more potential transplant donors for a recipient in need of a transplant, the kit comprising: a) one or more nucleic acid reagents to prepare a nucleic acid library from a nucleic acid sample; and b) one or more oligonucleotide probes that hybridise to gene target sequences of the nucleic acid sample.

The present disclosure also provides a kit for reducing the likelihood of a transplant recipient developing graft versus host disease, the kit comprising: a) one or more nucleic acid reagents to prepare a nucleic acid library from a nucleic acid sample; and b) one or more oligonucleotide probes that hybridise to gene target sequences of the nucleic acid sample.

In one embodiment, the gene target sequences are sequences for a highly polymorphic gene complex. The polymorphic gene complex may be a polymorphic gene complex pertaining to transplantation. In one embodiment, the polymorphic gene complex is an HLA gene complex. In other embodiments, the polymorphic gene complex is any other polymorphic gene complex. In another embodiment, the gene target sequences are sequences are sequences for any gene complex relating to a transplant whose transplant phenotype is based on gene or sequence copy number differences.

The present disclosure provides a kit using the methods disclosed herein for identifying one or more potential transplant donors for a recipient in need of a transplant, the kit comprising:

a) one or more nucleic acid reagents to prepare a nucleic acid library from a nucleic acid sample; and
b) one or more oligonucleotide probes that hybridise to gene target sequences of the nucleic acid sample.

The present disclosure also provides a kit using the methods disclosed herein for reducing the likelihood of a transplant recipient developing graft versus host disease, the kit comprising:

a) one or more nucleic acid reagents to prepare a nucleic acid library from a nucleic acid sample; and
b) one or more oligonucleotide probes that hybridise to gene target sequences of the nucleic acid sample.

The present disclosure provides use of a kit according to the methods disclosed herein for:
a) identifying one or more potential transplant donors for a recipient in need of a transplant;
b) reducing the likelihood of a transplant recipient developing graft versus host disease between one or more potential transplant donors for a recipient in need of a transplant;
c) reducing the likelihood of a transplant recipient developing graft versus host disease (GVHD) between one or more potential transplant donors for a recipient in need of a transplant; and
d) analysing sequences to identify one or more potential transplant donors for a recipient in need of a transplant.

The present disclosure provides a kit using the methods disclosed herein for:
a) identifying one or more potential transplant donors for a recipient in need of a transplant;
b) reducing the likelihood of a transplant recipient developing graft versus host disease between one or more potential transplant donors for a recipient in need of a transplant;
c) reducing the likelihood of a transplant recipient developing graft versus host disease (GVHD) between one or more potential transplant donors for a recipient in need of a transplant; and
d) analysing sequences to identify one or more potential transplant donors for a recipient in need of a transplant.

The kit may be used with a nucleic acid sample where the nucleic acid sample is genomic DNA. In one embodiment, the kit comprises one or more nucleic acid reagents to prepare a nucleic acid library comprises one or more reagents to bind to the genomic DNA, one or more reagents to fragment the genomic DNA and one or more reagents to tag the genomic DNA to beads.

In one embodiment, the kit contains oligonucleotide probes that comprises a capture tag, such as for example, the capture tag being biotin or streptavidin. The kit further comprises a binding agent, such as for example, the binging agent being biotin or streptavidin. The binding agent is coupled to a substrate such as, for example, the binding agent being a substrate or a bead. In one embodiment, the substrate or bead may be a magnetic substrate or magnetic bead.

The present disclosure provides a kit further comprising one or more nucleic acid reagents to perform sequencing of the nucleic acid library using the methods the methods disclosed herein wherein sequencing reads are generated in a computer readable form. In one embodiment, the generated sequencing reads are next generation sequencing (NGS) reads.

The present disclosure provides a kit that may further comprise a computer program to analyse and edit the NGS reads and generate a gene dosage map for each locus of a gene complex using the methods disclosed herein, wherein one or more potential transplant donors is identified as a transplant match and/or best transplant match for a recipient in need of a transplant. In one embodiment, the computer program is a sequence editing and alignment program. In on embodiment, the sequence editing and alignment program may be the TruSight HLA Assign™ 2.1 program and/or AlloSeq Assign software.

### Examples

### Example 1: DNA Library Preparation

DNA libraries were prepared from 100 ng genomic DNA using Illumina's commercially available ‘Illumina DNA Prep with Enrichment’ kits (formerly known as ‘Nextera Flex for Enrichment’ protocol), selecting for target inserts of 550 bp in size (Cat. No. 20025523 and 20025524). The protocol can be found in Illumina's ‘Ilumina DNA Prep with Enrichment Reference Guide’ (Document #1000000048041 v05, published June 2020) which can be downloaded at: https://support.illumina.com/content/dam/illumina-support/documents/documetation/chemistry_documentation/illumina_prep/illumina-dna-prep-with-enrichment-reference-1000000048041-05.pdf. This methodology is incorporated herein by reference.

All samples are clinical samples derived from hospital patients.

### Example 2: HLA Capture Using Intron-Specific Probes

HLA capture using intron-specific probes was performed using the methodology as described in WO 2015/085350 herein incorporated by reference.

### Example 3: Sequencing of Hybridized DNA

The amplified hybridized sample was sequenced on a MiSeq, iSeq or MiniSeq using Illumina 2×300 bp sequencing protocol. Sequencing reads are produced in deconvoluted (de-indexed) patient-specific sequence reads in the form of FASTQ files.

### Example 4: Results and Assignment of Sequences

The sequence data generated was analysed using the Assign™ TruSight version 2.1 software by (“Assign software”) by CareDx Inc. and/or AlloSeq Assign software which assists with the assignment of a human leukocyte antigen (HLA) type. The software analyses sequencing data from a library or libraries prepared with using the Illumina's commercially available ‘Illumina DNA Prep with Enrichment’ kit and protocol (formerly known as ‘Nextera Flex for Enrichment’ protocol). The Assign software by CareDx is commercially available from CareDx via purchase of CareDx's Trusight HLA typing kits: https://labproducts.caredx.com/products/trusight-hla/typing-kits/.

The Assign software may be downloaded at: https://labproducts.caredx.com/software/assign/assign-trusight/assign-trusight-v2-1. The operating manual ‘TruSight HLA Assign 2.1 RUO Software Guide’ (ILLUMINA PROPRIETARY Document #1000000010450 v01, published October 2016) is available at: https://labproducts.caredx.com/software/assign/assign-trusight/assign-trusight-v2-1/manuals/

Another software used is the AlloSeq Assign software by CareDx. The AlloSeq Assign software is commercially available from CareDx via purchase of CareDx's AlloSeq Tx 17 kit: https://labproducts.caredx.com/products/alloseq-hla/.

Using the Assign software program, the raw sequence data in FASTQ file format are imported into the Assign software. In one embodiment, the sequences are gene sequences. In another embodiment, the sequences are intergenic sequences. In another embodiment, the sequences are gene sequences and intergenic sequences. Base calling is performed and sequence editing is performed on the imported sequences. The consensus region of the edited sequences is compared with a reference genome, which consists of a sequence library of all known HLA alleles (HLA variants and motifs) as listed in the publicly available IMGT/HLA database.

The Assign software is calibrated by the inventors to analyse the imported and edited sequences and recognise specific segments of sequences by their polymorphic motifs in comparison with equivalent polymorphic motifs of the library of known HLA alleles.

The Assign software may be calibrated by the inventors to analyse the entire length of the sequences. It will be understood that the entire length of the sequences comprises various segments of sequences relating to one or more polymorphic motifs and comprises various segments of sequences relating to one or more non-polymorphic motifs.

Depending on the purposes and interest of the user, the Assign software may be calibrated to analyse only certain segments of the sequences of interest where the segments of sequences may contain one or more particular polymorphic motifs of interest. Analysis of particular segments of sequences relating to the one or more particular polymorphic motifs of interest involve comparing the one or more motifs of the imported sequences with equivalent one or more motifs of the HLA library of known HLA alleles. The Assign software may be calibrated by the inventors to align either the entire NGS sequences or certain segments of the NGS sequences containing one or more polymorphic motifs of interest in accordance to successively increasingly polymorphic loci and how the Assign software interprets insertions and deletions within the reads.

This enables the sequences (either entire sequences and/or segments of sequences relating to one or more motifs) to be assigned into the correct gene specific allocations and are termed “assigned reads”. Depending on the level of stringency desired, assignments of reads to each HLA gene may be based on any one of or all of the following criteria: regions of each locus; such as core exons; all exons; and/or entire sequences. Other reads to the exception (either entire sequences and/or segments of sequences relating to one or more motifs), which, for example, have a consensus region that does not align and/or have a sequence with inconsistent bases with about less than 80% sequence homology, when compared to the reference genome being the HLA allele library, are termed as “unassigned reads”. Other reads to the exception (either entire sequences and/or segments of sequences relating to one or more motifs) which, for example, have a consensus region that does align and/or have a sequence with consistent bases with about 80% to about 100% sequence homology when compared to the reference genome being HLA allele library, may still be termed as “unassigned reads” if the one or more polymorphic motifs of interest are found to have homology to more than one locus.

Entire NGS sequences and/or segments of NGS segments containing one or more motifs that have about 80% to about 100% sequence homology to the reference sequences genome of HLA alleles may be termed as “assigned reads” if the one or more motifs of interest are homologous to only one locus. Entire NGS sequences and/or segments of NGS segments containing one or more motifs that have about 80% to about 100% sequence homology to the reference sequences genome of HLA alleles may be termed as “unassigned reads” if the one or more motifs of interest are homologous to more than one locus.

If one or more motifs of interest present in entire sequences and/or segments of sequences are homologous to more than one locus and are designated by the Assign software to be “unassigned reads”, a user may choose to investigate other one or more motifs that may be present in said entire sequences and/or segments of sequences.

Unassigned reads are not allocated by the Assign software into HLA gene specific allocations. This is figuratively exemplified in FIGS. 1 and 2.

As shown in FIG. 1, the Assign software interrogates total hybrid-capture NGS reads or total HLA reads for all HLA genomic regions of interest which have been hybridized to by HLA target-specific biotinylated oligonucleotide probes in a first patient i.e. patient 1, which generated a total of 250,000 reads. Of the total 250,000 reads, these reads are analysed, edited and compared to a reference genome (i.e. a stored library of known sequences of HLA alleles). The consensus regions of the total reads are analysed and assigned by the Assign software into HLA gene specific allocations, namely, Gene A (with 27,000 assigned reads), Gene B (with 25,000 assigned reads) and Gene C (with 30,000 assigned reads) respectively.

FIG. 2 shows all sequence reads for HLA genomic regions (loci) of interest which have been hybridized to by HLA target-specific biotinylated oligonucleotide probes in a second patient i.e. patient 2, which generated a total of 220,000 reads. Of the total 220,000 reads for patient 2, there are 24,000 assigned reads for Gene A, 11,000 assigned reads for Gene B and 26,000 assigned reads for Gene C.

Owing to high polymorphism of HLA genes and inheritance of the entire MHC as an HLA haplotype in a Mendelian fashion from each parent, a mixed population (non-endogamic) will not have two individuals with exactly the same set of HLA genes and molecules, with the exception of identical twins. Accordingly, as exemplified in FIGS. 1 and 2, patient 1 and patient 2 will not have the same number of total HLA sequence reads and will therefore also have differing numbers of assigned reads for genes A, B and C.

### Example 5: Generation of Gene Dosage Map from Assigned Reads

The assigned reads allocated by the Assign software and/or AlloSeq Assign software was used to compare the amount of sequence reads for a patient sample with another patient, at a given locus. This is exemplified in FIG. 3 with for example, the HLA-H gene.

In order to compare the amount of sequence reads for a patient sample, at a given locus, it is crucial that compared reads are relative to total aligned (assigned) sequence reads. In FIG. 3, column A denotes samples from twenty different patients. Column B denotes the total NGS reads for each patient. Column C denotes the assigned reads for all HLA genes and column D denotes assigned reads specifically to the HLA-H gene. Owing to the high degree of polymorphism in HLA genes, no two individuals will have the same number of total reads, assigned reads and HLA-H specific reads as shown in FIGS. 3 and 4. As shown in FIG. 4, several individuals (denoted by arrows) are seen to have a reduction in sequence reads for the HLA-H locus compared to total sequence reads, which may be more overtly demonstrated via a ratio of the two measures (see column F of FIG. 3 and FIG. 5).

HLA-H read count is relative to the total assigned read count and must be normalized before being compared to another individual, whose total read count likely differs. To normalise sequence data, for a given locus, the locus-specific sequence reads for a patient sample are divided by that patient's total assigned reads. The resulting patient's proportion of sequence reads, may easily be compared to other patient samples in the form of a ratio of the mean proportions. In FIG. 3, by dividing the gene specific HLA-H reads in column D by the total sequence reads assigned to loci (Assigned sequence reads in column C), it is possible to derive a locus-specific proportion of reads for each individual or patient. In order to best compare the proportion of sequence reads, it is possible to divide the proportion of reads for an individual by the mean proportion of sequence reads for two copy individuals (in most cases all individuals). This results in a ratio of gene dose (column F of FIG. 3), which may be expressed as a percentage proportion where differences between gene loci are easily demonstrated (FIG. 2).

To normalise sequence data, for a given locus, the locus-specific sequence reads for a patient sample are divided by that patient's total assigned reads. The resulting patient's proportion of sequence reads, may easily be compared to other patient samples in the form of a ratio of the mean proportion. Table 1 illustrates raw values for total assigned sequence read count and HLA-H-specific sequence read count for twenty patient samples. By dividing the gene specific (HLA-H) reads by the total sequence reads assigned to loci (Assigned sequence reads), it is possible to derive a locus-specific proportion of reads for each individual (Table 2). In order to best compare the proportion of sequence reads, it is possible to divide the proportion of reads for an individual by the mean proportion of sequence reads for two copy individuals (in most cases all individuals). This results in a ratio of gene dose (Table 3), which may be expressed as a percentage proportion where differences are easily demonstrated and visualised (FIG. 5). This means that a percentage of about 100 percent equates to a gene copy number of 2 in a sample or patient, a percentage of about 50 percent equates to a gene copy number of 1 in a sample or patient and a percentage of about 0 percent equates to a gene copy number of zero in a sample or patient. The results from FIG. 3 are plotted into the histogram of FIG. 5. As shown in FIG. 5, patients 2, 7, 8, 11, 17, 22 and 24 all possess only one copy of the HLA-H gene, a result that could not be demonstrated definitively via nucleotide sequencing.

By repeating the locus-specific analysis for all contiguous loci, for a given patient, it is possible to generate a map of gene dosage for all HLA genes across the MHC gene block or complex, as shown in FIGS. 6 to 8. FIGS. 6 to 8 show the generated map of gene dosage based on the locus-specific analysis technique of the present disclosure for three individuals or patients. The generated gene dosage map is a pictorial showing the relative gene dosage amounts of each and every locus within the gene complex relative to each other. The relative amounts of each and every locus is the copy number of each and every locus of a gene complex relative to each other. The more similar a gene dosage map of a first individual when compared to a second individual, the higher the probability of the first and second individual having a successful transplant outcome. The higher the correlation of a gene dosage map of a first individual when compared to a second individual, the higher the probability of the first and second individual having a successful transplant outcome.

The gene dosage map can be compared amongst different individuals or patients. Similarity, or higher correlation, of gene dosage map data can be used for more improved diagnosis or prognosis of tissue or organ transplant matching between a donor and recipient. From FIGS. 6 to 8, gene copy number difference measured may be 0 or may be 1 or may be 2. The gene copy number difference measured may be 3 or may be more than 3.

FIG. 9 is a graphical representation of the percentage proportion of HLA genes: HLA-A; HLA-B and HLA-C in 18 samples, whereby the sequences were gene rated using PCR-based methodology and not using the hybrid-capture NGS sequencing technique of the present disclosure. The percentage proportion for each of the HLA-genes was calculated using the method disclosed in the present disclosure. Sequences generated using PCR-based methodology is not an ideal method for determining gene dosage because exponential propagation of DNA from a sample will result in decreased uniformity between loci and patient samples. In the present disclosure, the use of hybrid-capture NGS technique allows for comparison using the same concentrations of DNA and the sequence reads can be adjusted using total sequence reads.

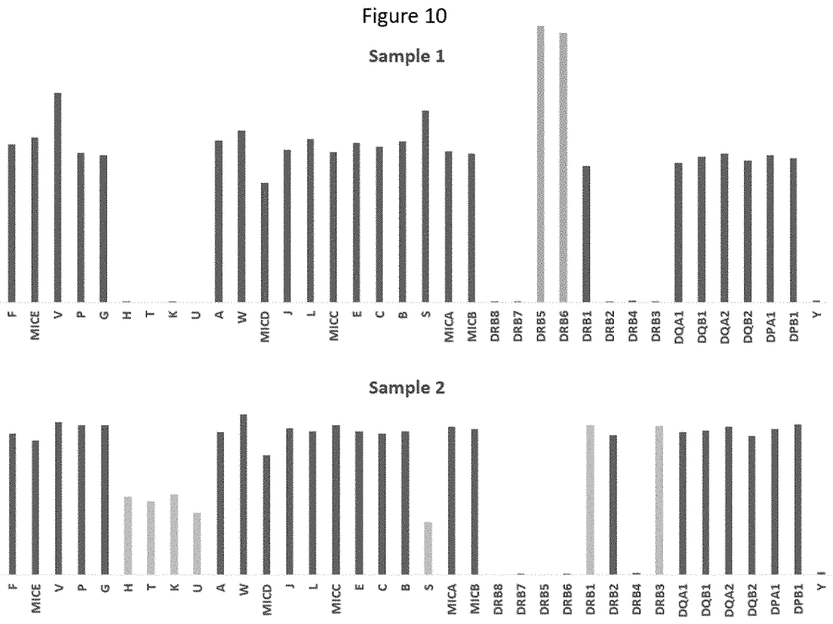

FIG. 10 shows the gene dosage map generated via the method of the present disclosure for a donor-recipient pairing likely resulting in poor transplant outcomes. As shown in FIG. 10, the generated gene dosage map informs that the gene content of the two individuals are different.

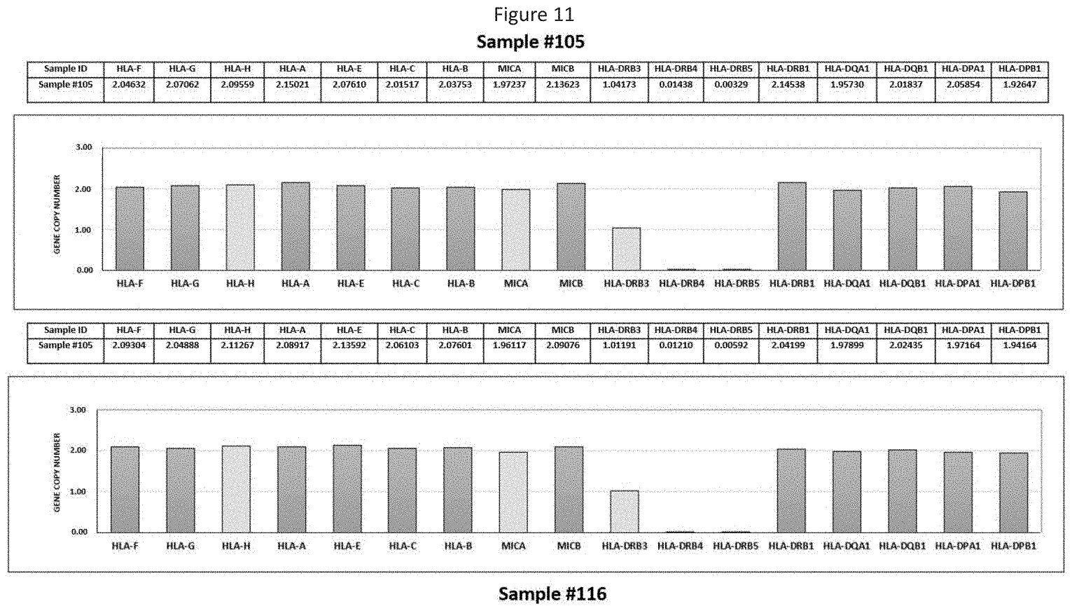

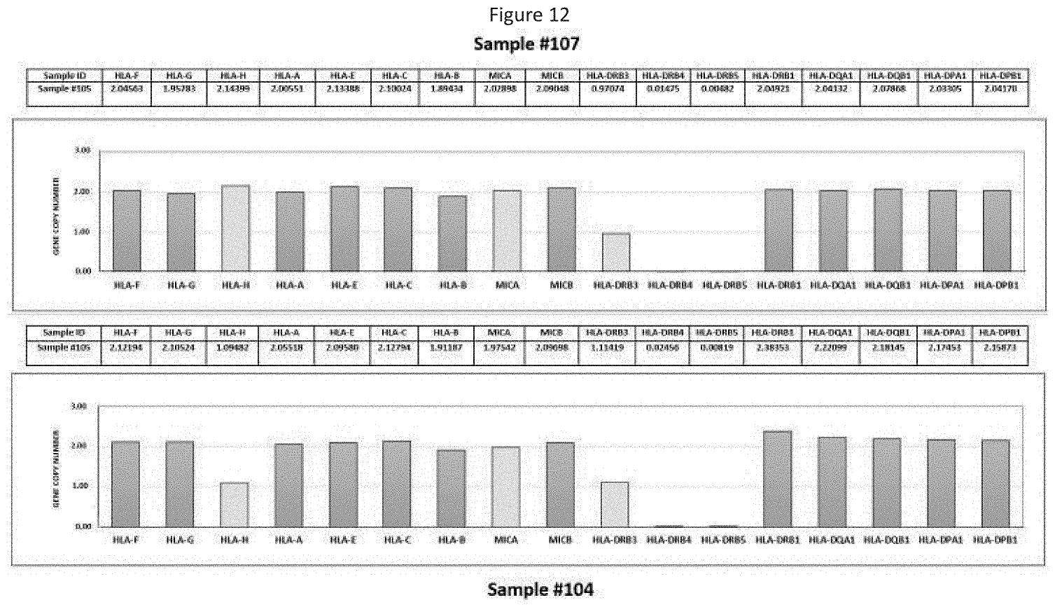

FIGS. 11 and 12 shows the gene dosage map generated via the method of the present disclosure for a first pair of clinical samples: samples #105 and #116, and a second pair of clinical samples: samples #107 and #104, respectively. As shown in FIGS. 11 and 12, the generated gene dosage map informs that the gene content of these two clinical sample pairings are very similar.

The data in the present disclosure demonstrates that the use of gene dosage maps generated from the use of the locus-specific analysis technique on NGS data of the present disclosure enables improved diagnosis as well as prognosis of tissue and organ transplant outcomes between a donor and recipient. The present disclosure enables improved diagnosis as well as prognosis of tissue and organ transplant outcomes between a donor and recipient relating to graft versus host disease (GVHD) or any transplant where transplant phenotype is observed based on sequence and/or gene copy number differences following transplantation of a graft or organ from the one or more transplant donors.

It will be appreciated by the person skilled in the art that numerous variations and/or modifications may be made to the invention as shown in the specific embodiments without departing from the scope of the invention as broadly described. The present embodiments are therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Garcia, M. A., Yebra, B. G., Flores, A. L. L., Guerra, E. G. (2012) “The major histocompatibility complex in transplantation”, J Transplan. 20:842141.
Sheldon, S. and Poulton, K. (2006) “HLA typing and its influence on organ transplantation” Methods Mol Biol. 333:157-74.
Guild W R, Harrison J H, Merrill J P, Murray J. (1955) “Successful homotransplantation of the kidney in an identical twin”, Transactions of the American Clinical and Climatological Association; 67:167-173.
Klein J A N, Sato A. (2000) “The HLA system: first of two parts”, N Engl J Med. 343(10):702-709. doi: 10.1056/NEJM200009073431006.
Mandi, B. M (2013) “A glow of HLA typing in organ transplantation” Clin Transl Med. 2013 Feb. 23; 2(1):6. doi: 10.1186/2001-1326-2-6.

1.-56. (canceled)

57. A computer-implemented method for identifying one or more potential transplant donors for a recipient in need of a transplant, the method comprising:
a) generating a gene dosage map, using a computer, for each locus of a gene complex for the one or more potential donors and the recipient based on loci-assigned sequences;
b) comparing the gene dosage maps of the one or more potential donors and the recipient, using a computer; and
c) determining one or more transplant donors as a transplant match for a recipient in need of a transplant if the gene dosage map of the one or more transplant donors correlates with the gene dosage map of the recipient in need of a transplant, using a computer;
wherein the closer the correlation between the gene dosage maps of the one or more donors compared to the recipient, the higher the probability of the one or more donors being a transplant match and/or best transplant match for the recipient.

58. A computer-implemented method for identifying one or more potential transplant donors for a recipient in need of a transplant, the method comprising:
a) generating sequences of a gene complex, using a computer, from a nucleic acid sample obtained from the one or more potential transplant donors and the recipient;
b) assigning a plurality of the sequences generated in step (a) corresponding to each locus of the gene complex, using a computer;
c) determining gene dosage for each locus of the gene complex from the plurality of sequences assigned in step (b), using a computer;
d) generating a gene dosage map of the gene complex for the one or more potential transplant donors and the recipient from the gene dosage for each of the locus of the gene complex determined in step (c), using a computer; and
e) comparing the generated gene dosage map of the one or more potential transplant donors with the generated gene dosage map of the recipient, using a computer;
wherein the one or more potential transplant donors is identified as a transplant match and/or best transplant match for a recipient in need of a transplant if the gene dosage map of the one or more transplant donors correlates with the gene dosage map of the recipient.

59. A computer-implemented method for reducing the likelihood of a transplant recipient developing graft versus host disease (GVHD), the method comprising:
a) generating sequences of a gene complex, using a computer, from a nucleic acid sample obtained from the one or more potential transplant donors and the recipient;
b) assigning a plurality of the sequences generated in step (a) corresponding to each locus of the gene complex, using a computer;
c) determining gene dosage for each locus of the gene complex from the plurality of sequences assigned in step (b), using a computer;
d) generating a gene dosage map of the gene complex for the one or more potential transplant donors and the recipient from the gene dosage for each locus of the gene complex determined in step (c), using a computer; and
e) comparing the generated gene dosage map of the one or more potential transplant donors with the generated gene dosage map of the recipient, using a computer;
wherein the gene dosage map of the one or more potential transplant donors correlating with the gene dosage map of the recipient in need of a transplant is indicative of reduced likelihood of the transplant recipient developing graft versus host disease following transplantation of a graft from the one or more transplant donors.

60. A computer-implemented method for analysing sequences to identify one or more potential transplant donors for a recipient in need of a transplant, the method comprising:
a) generating sequences of a gene complex, using a computer, from a nucleic acid sample obtained from the one or more potential transplant donors and the recipient;
b) assigning a plurality of the sequences generated in step (a) corresponding to each locus of the gene complex, using a computer;
c) determining gene dosage for each locus of the gene complex from the plurality of sequences assigned in step (b), using a computer;
d) generating a gene dosage map of the gene complex for the one or more potential transplant donors and the recipient from the gene dosage for each of the locus of the gene complex determined in step (c), using a computer; and
e) comparing the generated gene dosage map of the one or more potential transplant donors with the generated gene dosage map of the recipient, using a computer;
wherein the one or more potential transplant donors is identified as a transplant match and/or best transplant match for a recipient in need of a transplant, if the gene dosage map of the one or more transplant donors correlates with the gene dosage map of the recipient.

61. A computer-implemented method of preventing graft versus host disease (GVHD) disease between one or more potential transplant donors and a recipient comprising
a) generating sequences of a gene complex, using a computer, from a nucleic acid sample obtained from the one or more potential transplant donors and the recipient;
b) assigning a plurality of the sequences generated in step (a) corresponding to each locus of the gene complex, using a computer;
c) determining gene dosage for each locus of the gene complex from the plurality of sequences assigned in step (b), using a computer;
d) generating a gene dosage map of the gene complex for the one or more potential transplant donors and the recipient from the gene dosage for each locus of the gene complex determined in step (c), using a computer; and
e) comparing the generated gene dosage map of the one or more potential transplant donors with the generated gene dosage map of the recipient, using a computer;
wherein the gene dosage map of the one or more potential transplant donors correlating with the gene dosage map of the recipient in need of a transplant is indicative of reduced likelihood of the transplant recipient developing graft versus host disease following transplantation of a graft and/or tissue and/or organ from the one or more transplant donors, and
selecting graft and/or tissue and/or organ from a transplant donor having a gene dosage map that correlates with the gene dosage map of the recipient for transplant to the recipient.

62. The computer-implemented method of claim 58, wherein generating the gene dosage map for each locus of the gene complex for the one or more potential donors and the recipient comprises dividing the plurality of sequences assigned to each locus by the plurality of sequences assigned to all loci of the gene complex.

63. The computer-implemented method of claim 57, wherein the gene dosage for each locus is copy number for each locus, or all loci, of the gene complex.

64. The computer-implemented method of claim 63, wherein the copy number for each locus and all loci of the gene complex allows determination of zygosity for each locus and all loci of the gene complex.

65. The computer-implemented method of claim 63, wherein the copy number of each locus and all loci of the gene complex allows determination of whether two alleles have an identical sequence.

66. The computer-implemented method of claim 57, wherein the gene complex is a highly polymorphic gene complex.

67. The computer-implemented method of claim 57, wherein the gene complex is a gene complex pertaining to transplantation.

68. The computer-implemented method of claim 57, wherein the gene complex is an HLA gene complex.

69. The computer-implemented method of claim 58, wherein the step (b) of assigning a plurality of the sequences generated in step (a) corresponding to each locus of the gene complex is based on: one or more regions of each locus; all exons in each locus; and/or an entire sequence of each locus.

70. The computer-implemented method of claim 58, wherein step (b) comprises assigning a plurality of the sequences generated in step (a) using a computer program.

71. The computer-implemented method of claim 70, wherein the computer program is a sequence editing and alignment program.

72. The computer-implemented method of claim 58, wherein the step of a) generating sequences of a gene complex, using a computer, from a nucleic acid sample obtained from the one or more potential transplant donors and the recipient comprises:
a) contacting a nucleic acid sample from the one or more transplant donors and the recipient with oligonucleotide probes, wherein the oligonucleotide probes hybridize togene target sequences in the nucleic acid sample;
b) enriching a nucleic acid by hybridizing the nucleic acid to one or more oligonucleotide probes;
c) separating nucleic acid hybridized to the one or more oligonucleotide probes from nucleic acid not hybridized to the one or more oligonucleotide probes; and
d) sequencing the enriched nucleic acid to identify one or more gene alleles;
wherein the gene target sequences are in a non-coding region of the gene.

73. The computer-implemented method of claim 72, wherein the gene complex is a highly polymorphic gene complex.

74. The computer-implemented method of claim 72, wherein the gene complex is a gene complex pertaining to transplantation.

75. The computer-implemented method of claim 72, wherein the gene complex is an HLA gene complex.

76. The computer-implemented method of claim 72, wherein the method comprises amplifying the nucleic acid bound to the one or more oligonucleotide probes.

77. The computer-implemented method of claim 72, wherein the method comprises sequencing an HLA gene exon, or a gene exon pertaining to transplantation.

78. The computer-implemented method of claim 72, wherein the method comprises sequencing an entire HLA gene complex, or any entire gene complex pertaining to transplantation.

79. The computer-implemented method of claim 72, wherein the one or more oligonucleotide probes comprises a capture tag.

80. The computer-implemented method of claim 79, wherein the capture tag is biotin or streptavidin.

81. The computer-implemented method of claim 79, wherein the method further comprises contacting the capture tag with a binding agent.

82. The computer-implemented method of claim 81, wherein the binding agent is biotin or streptavidin.

83. The computer-implemented method of claim 72, wherein the nucleic acid sample from the one or more transplant donors and the recipient in need of a transplant that is contacted with the one or more oligonucleotide probes comprises single stranded nucleic acid.

84. The computer-implemented method of claim 72, wherein the nucleic acid sample is fragmented before or after being contacted with the one or more oligonucleotide probes.

85. The computer-implemented method of claim 84, wherein the fragments of the nucleic acid sample have an average length greater than about 100 bp.

86. The computer-implemented method of claim 57, wherein the nucleic acid sample from the one or more transplant donors and the recipient in need of a transplant is genomic DNA extracted from a biological sample.

87. The computer-implemented method of claim 86, wherein the biological sample is whole blood.

88. The computer-implemented method of claim 86, wherein the genomic DNA is at a concentration of about 10 ng/μl to about 100 ng/μl.

89. The computer-implemented method of claim 72, wherein sequencing is performed using high-throughput sequencing.

90. The computer-implemented method of claim 89, wherein the high-throughput sequencing is hybrid-capture next generation sequencing.

91. The computer-implemented method of claim 58, wherein the sequences are generated in a computer readable form.

92. The computer-implemented method of claim 91, wherein the computer readable form is FASTQ.
